(19) United States
(12) Patent Application Publication
(10) Pub. No.: US 2013/0273096 A1
(43) Pub. Date: Oct. 17, 2013

(54) Therapeutic Sulfated Polysaccharides, Compositions Thereof, and Methods for Treating Patients

(71) Applicant: Bruce A. Daniels, Oklahoma City, OK (US)

(72) Inventor: Bruce A. Daniels, Oklahoma City, OK (US)

(21) Appl. No.: 13/646,496

(22) Filed: Oct. 5, 2012

Related U.S. Application Data
(63) Continuation-in-part of application No. 13/624,742, filed on Sep. 21, 2012.
(60) Provisional application No. 61/543,684, filed on Oct. 5, 2011.
(60) Provisional application No. 61/543,684, filed on Oct. 5, 2011.

(51) Int. Cl. A61K 31/737; G01N 33/50

(52) U.S. Cl. CPC A61K 31/737; G01N 33/5008
USPC 424/195.17; 536/118; 435/29; 514/54

(57) ABSTRACT

Disclosed are methods and compositions for the treatment of a variety of disorders in subjects by affecting the glycocalyx of a subject in need of such treatment. The methods comprise administering to a subject an effective amount of a sulfated polysaccharide (SP) or analogue thereof, the SP being a non-animal based (e.g., plant, or bacteria derived) sulfated polysaccharide. The SPs can be administered as single agents, or in combination with one another, or with other medications to promote efficacy. Pharmaceutical compositions and comestibles including such SPs are also described.

### CROSS REFERENCE TO RELATED APPLICATIONS

This application is a continuation-in-part application of pending U.S. patent application Ser. No. 13/624,742, filed Sep. 21, 2012, which claims priority to U.S. Provisional patent application Ser. No. 61/537,558, filed Sep. 21, 2011, and U.S. Provisional patent application Ser. No. 61/543,684, filed Oct. 5, 2011, all of which are incorporated herein by reference in their entirety.

### STATEMENT REGARDING FEDERALLY SPONSORED RESEARCH OR DEVELOPMENT

Not applicable.

### REFERENCE TO APPENDIX

Not applicable.

### BACKGROUND OF THE INVENTION

1. Field of the Invention

The inventions disclosed and taught herein relate generally to sulfated polysaccharides and their therapeutic uses in treating disorders within subjects. In particular, the invention relates to sulfated polysaccharides from non-animal sources for use in the therapeutic treatment of subjects suffering from disorders associated with the glycocalyx, metabolic syndromes, vascular diseases and disorders, or sickle-cell anemia, and in need of such therapeutic treatment.

2. Description of the Related Art

One of the most studied marine sulfated polysaccharides are the sulfated galactans (SGs). Together with sulfated fucans, the SGs are, after glycosaminoglycans, the most widely studied sulfated polysaccharides worldwide. In general, the SGs are polymers of α-L- and/or α-D- or β-D-galactopyranosyl units [Pomin, V. P., et al., Glycobiology, Vol. 18, pp. 1016-1027 (2008)]. The structures of these strongly anionic macromolecules vary among species, although their main structural features are conserved among phyla. They usually show high molecular weights (MWs) (≧100 kDa). These glycans possess highly electronegative charge density from their sulfated esters which allows them electrostatic interactions with specific proteins, triggering in consequence their biological effects. However, these intermolecular interactions have been usually stereospecific dependent rather than a mere or simple consequence of charge interactions [Pomin, V. P., Biopolymers, Vol. 91, pp. 601-609 (2009)].

These compounds are essentially isolated from marine organisms such as macroalgae (brown, green, and red), and certain invertebrates like echinoderms (sea cucumber and sea urchins) or tunicates (ascidians). In general, these polymers are mainly composed of fucopyranosyl units exclusively in their α-L-form, or of α-L- and/or α-D- or β-D-galactopyranosyl units. The structures of these strongly anionic macromolecules vary among species, although their main structural features are conserved among phyla.

Until recently, sulfated fucans have been described only in brown algae (Phaeophyta), sea cucumber (Echinodermata, Holothuroidea), and sea urchins (Echinodermata, Echinoidea). A recent article reported at the first time a novel SG isolated from the sea grass Rupia maritima [Aquino, R. S., et al., Glycobiology, Vol. 15, pp. 11-20 (2005)]. The sea grass is a group of vascular flowering plants (angiosperms) which grow in highly saline marine environments. The structure of the sulfated D-galactan from R. maritime is composed of a regular tetrasaccharide repeating unit (FIG. 1e). Like red algae, the marine angiosperm polysaccharide contains both α- and β-D-galactosyl isomers; however, these units are not distributed in an alternating sequence, and the angiosperm molecule has much clearer and well-defined structure. The SG in this marine plant unequivocally contributes for the structural arrangement of the cell wall, but it also seems to be physiologically involved in the osmotic regulation of the epidermal cells, once this plant is ecologically allocated in habitats with high salinity variations. With few exceptions [see, e.g., Chevolot, L., et al., Carbohydrate Research, Vol. 319, pp. 154-165 (1999); Bilan, M. I., et al., Carbohydrate research, Vol. 337, pp. 719-730 (2002)], most of the structures found in brown algae are highly heterogeneous due to several sulfation and glycosylation sites; no clear evidence of repetitive units, and also the common presence of branching residues in any position. All these heterogeneities often make difficult the complete structural elucidation of the algal sulfated fucans. This intricate structural arrangement is directly related to the function of the brown algal sulfated fucans as a structural organizer component of the assembled molecules of the cell wall. This complex structure together with high molecular mass allows the highly complex organization of the cell wall through bonds with different types of molecules: peptides, alginic acid, pectin, cellulose, heteropolysaccharides, among others [Andrade, L. R., et al., J. Structural Biology, Vol. 145, pp. 216-225 (2004)].

In contrast, the structures of echinoderm-sulfated fucans are much simpler. These glycans are composed of well-defined repetitive units that allow the complete determination of their sulfation patterns, glycosidic linkage types, and anomeric configurations for all residues (see FIG. 1). These macromolecules have been isolated from the egg-jelly layer of the sea urchins, and the body wall of sea cucumbers. Different from the random structure of the brown algal sulfated fucans, clear structural patterns are required for invertebrate sulfated fucans due to their specific biological functions. In the case of sea-urchins, they are involved in a very rare case of carbohydrate-induced signal transduction, the acrosome reaction (AR). The characteristic regular structures of each sea urchin species appear to be important to maintain the intra-specificity found in their external fertilization [Vilela-Silva, A.-C., et al., Int. J. Dev. Biol., Vol. 52, pp. 551-559 (2008)].

The sulfated galactans have also be isolated from the cell wall of green (Clorophyta) or of red (Rhodophyta) algae. Similarly to the sulfated fucans, the sulfated galactans can be found in the egg jelly coats of a few sea urchin species, and have been reported to have been isolated from the tunics of ascidians (Urochordata, Ascidiacea) [Santos, J. A., et al., European J. Biochem., Vol. 204, pp. 669-677 (1992)]. There was also a recent description of a sulfated galactan isolated from a sea grass, a marine angiosperm [Aquino, R. S., et al., Glycobiology, Vol. 15, pp. 11-20 (2005)]. The sulfated galactans from invertebrates (see FIG. 2) and the marine angiosperm show the same structural pattern of simple and well-defined units as found in invertebrate sulfated fucans (see FIG. 1). The structures of green algal sulfated galactans are however more heterogeneous, but simpler than brown algal sulfated fucans. The green algal macromolecules are predominantly composed of 3-β-D-Galp, but without a regular or repetitive unit, and with a heterogeneous distribution of sulfation (however, mainly 4- and/or 6-sulfate). The red algal sulfated galactans have a very regular backbone, composed always of disaccharide repeating units, but are also highly heterogeneous in their sulfation patterns which vary from species to species (see, FIG. 2D).

Although algal homopolysaccharides exhibit potent pharmacological actions, their structural complexities and/or partial characterization do not allow a complete understanding of their biochemical properties. It is usually hard to establish the structure-function relationship for algal polysaccharides, especially sulfated fucans. On the other hand, it is dearer to understand the biological actions of invertebrate polysaccharides because of their well-defined structures. Therefore, the invertebrate homopolysaccharides have been preferentially chosen for subsequent pharmacological studies as potential drug candidates [See, Mourao, P.A., Current Pharmaceutical Design, Vol. 10, pp. 967-981 (2004)]. It is believed, although it has not been shown, that the main structural features of the polysaccharides (sugar type, sulfation and glycosylation sites, and orientational binding preferences) can specifically account for the exhibition of favorable biological actions in therapeutic applications.

As a class, however, when viewed from a therapeutic and potential drug standpoint, sulfated polysaccharides are characterized by a plethora of biological activities with often favorable tolerability profiles in animals and humans. These polyanionic molecules are often derived from animal tissues and encompass a broad range of subclasses including heparins, glycosaminoglycans, fucoidans, carrageenans, pentosan polysulfates, and dermatan or dextran sulfates [see, for example, Toida, et al. (2003), Trends in Glycoscience and Glycotechnology 15:29-46)]. Lower molecular weight, less heterogeneous, and chemically synthesized sulfated polysaccharides have also been reported and have reached various stages of drug development in recent years [see, for example, (Sinay, (1999), Nature, Vol. 398, pp. 377-378; Orgueira, et al. (2003), Chemistry, Vol. 9, pp. 140-169; and, Williams, et al. (1998), Gen. Pharmacol., Vol. 30, pp. 337-341]. Heparin-like sulfated polysaccharides exhibit differential anticoagulant activity mediated through antithrombin III and/or heparin cofactor II interactions [Toida et al., supra]. Notably, certain compounds, of natural origin or chemically modified, exhibit other biological activities at concentrations (or doses) at which inhibitory activity is not substantial [See, for example, Wan, et al. (2002), Inflamm. Res., Vol. 51, pp. 435-443; Bourin, et al. (1993), Biochem. J., Vol. 289 (Pt 2), pp. 313-330; and, Luyt, et al. (2003), J. Pharmacol. Exp. Ther., Vol. 305, pp. 24-30)].

The inventions disclosed and taught herein are directed to therapeutic compositions comprising sulfated polysaccharides which exhibit specific biological activity, and which are isolated from a variety of organisms, excluding animals, as well as methods of treatment using such compositions.

### BRIEF SUMMARY OF THE INVENTION

The novel feature of the present disclosure is that sulfated polysaccharides of non-animal, especially non-vertebrate animal, origin, have been found to have beneficial therapeutic effects on a variety of disease targets, including sickle cell anemia and other hemoglobin-type disorders, as well as disorders and maladies associated with the glycocalyx of the subject, metabolic disorders and diseases (metabolic syndromes), vascular diseases and disorders, and atherosclerotic disease.

In accordance with an embodiment of the present disclosure, methods of treating a glycocalyx-degradation associated disorder in a subject in need of such treatment are described, the method comprising administering an effective amount of pharmaceutical composition comprising a compound of formula (I),

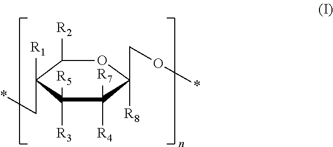

or a solvate, prodrug, hydrate, derivative, or analog thereof, wherein,
R₁, R₂, R₃, R₄, R₅, R₆, R₇ and R₈ are either the same or different,
at least one of R₁-R₈ is hydroxyl,
at least one of R₁-R₈ is OSO₃⁻M⁺, with M being selected from the group consisting of Na, K, Li, or H,
and the remaining residues are independently selected from the following groups consisting of hydroxyl, thiol, halo, nitro, cyano, alkyl, alkenyl, alkynyl, sulfonamido, amino acid, amino acid esters, amino acid amides, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, hydroxamic acid, tetrazolyl, carbohydrate, fucan, sorbitan, sugar, or alditol; and
n is an integer selected from 1 to 20,000, inclusive, and
wherein the points of chirality (*) within each residue may be alpha (α), beta (β), or alternating alpha and beta between residues.

In a further embodiment of the present disclosure, methods and compositions for treating a glycocalyx-related disorder in a patient are described, the method comprising the steps of: (a) administering an effective amount of a pharmaceutical composition comprising: (1) a pharmaceutically effective amount of a sulfated polysaccharide that is not of animal origin, or pharmaceutically acceptable salts, hydrates, solvates, or prodrugs thereof; and (2) a pharmaceutically acceptable carrier, wherein the administration of the pharmaceutical formulation positively acts upon the glycocalyx in the patient.

In accordance with a further embodiment of the present disclosure, methods of screening for a therapeutic agent for a glycocalyx-related disease or metabolic syndrome are described, the method of which comprises: a) contacting a test compound with cells expressing a heparin sulfate protein; b) measuring expression levels of the protein in the cells; and c) selecting a sulfated polysaccharide compound that increases the level of expression compared with that measured in the absence of the polysaccharide compound, thus selecting a therapeutic agent for the glycocalyx-related disease.

In accordance with a further aspect of the present disclosure, a method of suppressing glycocalyx degredation in endothelial cells in a region of a patient is described, the method comprising administering to the patient a composition comprising as an active ingredient a sulfated polysaccharide. In accordance with aspects of this embodiment, the method has an effect of suppressing angiogenesis in cardiac valves. In further aspects of this embodiment, the method has an effect of suppressing vascular damage in a patient with diabetic retinopathy.

In a further embodiment of the present disclosure, therapeutic composition for the treatment of a disorder in a subject selected from vascular disorders, small vessel disease or disorders, or rheumatoid arthritis are described, wherein the composition comprises a purified, sulfated polysaccharide at a therapeutic concentration, and wherein the composition is at a dose the provides a beneficial effect to an individual in need of treatment of one or more of the disorders. In accordance with aspects of this embodiment, the therapeutic composition comprises one or more naturally-occurring, non-animal derived (e.g., plant or bacteria based) sulfated polysaccharide in a therapeutically effective amount. In further accordance with aspects of this embodiment, the small vessel disease includes coronary microvascular disease.

In accordance with a further embodiment of the present disclosure, a method of treating a glycocalyx-associated disorder in a subject is described, the method comprising administering to a subject in need thereof a therapeutic amount of a composition comprising at least one purified, sulfated polysaccharide, sulfated polysaccharide polymer, or extract thereof, and wherein the composition is at a dose sufficient to displace heparans from at least one glycocalyx of an endothelium of the subject. In certain aspects of this embodiment, the sulfated polysaccharide is rhamnan sulfate, or a rhamnan sulfate isolate. In yet another aspect of this embodiment, the rhamnan sulfate or rhamnan sulfate isolate is derived from the species Monostroma nitidum.

In a further embodiment of the present disclosure, a therapeutic dosage form useful for the treatment of a vascular or vessel-related disorder in a subject is described, the dosage form comprising a sulfated polysaccharide, a sulfated polysaccharide isolate of a natural organism, or an extract or polymer extract of a natural sulfated polysaccharide.

In accordance with yet another embodiment of the present disclosure, a method of treating diabetic retinopathy in a subject is described, the method comprising administering a composition comprising a therapeutically effective amount of one or more naturally-occurring sulfated polysaccharides of formula I, II or III.

In accordance with a further embodiment of the present disclosure, methods for therapeutically reducing detrimental endothelial permeability in a living subject are described, the method comprising the step of administering an effective amount of a composition comprising one or more naturally-occurring sulfated polysaccharides to the living subject after occurrence of an increased endothelial permeability to fluids and protein in at least one region of the body of the subject.

In accordance with further aspects of the disclosure, the present invention provides compounds, compositions, kits, and methods comprising sulfated polysaccharide compounds and natural product extracts containing such compounds for the prevention and treatment of inflammatory and metabolic disorders and metabolic syndromes, in particular, insulin resistance syndromes, diabetes, obesity, weight gain, cardiovascular disease and cancer.

In accordance with yet another embodiment of the present disclosure, an oral composition for treating a metabolic syndrome or cardiovascular disease in a subject in need thereof is described, wherein the composition comprises a therapeutically effective amount of one or more naturally-occurring sulfated polysaccharides, and wherein the composition is in a dosage form for oral administration. In accordance with aspects of this embodiment, the metabolic syndrome can include insulin related disorders, diabetes, and/or obesity in a subject.

In accordance with a further embodiment of the present disclosure, a method for treating a metabolic syndrome in a human is described, the method comprising: administering a therapeutically effective amount of a non-animal derived sulfated polysaccharide to a human having metabolic syndrome, wherein a human has metabolic syndrome if the human has at least three characteristics selected from the group consisting of: (a) a waist circumference greater than 102 cm for men and greater than 88 cm for women; (b) a triglyceride level greater than 150 mg/dl; (c) an HDL-cholesterol level less than 40 mg/dl for men and less than 50 mg/dl for women; (d) a blood pressure greater than or equal to 130/85 mmHG; and (e) a fasting glucose level greater than 110 mg/dl, such that after the administering of the sulfated polysaccharide composition, the human no longer has at least three characteristics selected from the group consisting of (a), (b), (c), (d), and (e).

In accordance with a further embodiment of the present disclosure, methods and compositions for preventing and treating atherosclerosis and diseases related thereto are provided, as well as conditions associated with increased endothelial permeability. According to the invention, the administration of a composition comprising a sulfated polysaccharide, such as rhamnan sulfate or an analog or derivative thereof, may exhibit a beneficial effect in endothelial cells of a subject in need of such therapy, such as by controlling or regulating serum LDL levels that are known to be atherogenic.

### BRIEF DESCRIPTION OF THE SEVERAL VIEWS OF THE DRAWINGS

The following figures form part of the present specification and are included to further demonstrate certain aspects of the present invention. The invention may be better understood by reference to one or more of these figures in combination with the detailed description of specific embodiments presented herein.

FIG. 1 illustrates the chemical structures of the repeating units of sulfated a-L-fucans from the body wall of a sea cucumber (A), and from the egg jelly coat of sea-urchins (B-G). The species-specific structures vary in sulfation patterns (e.g., exclusively 2- and/or 4-positions), in glycosidic linkages.

FIG. 2 illustrates the chemical structures of the repeating units of the sulfated galactans from the egg jelly coat of a sea urchin (A), from the tunic of ascidians (B and C), and from red algae (D).

FIG. 3A illustrates a general chemical representation of Rhamnan sulfate.

FIG. 3B illustrates a further general chemical representation of Rhamnan sulfate.

FIG. 4 illustrates an exemplary disaccharide compositional analysis of EC (embryonal carcinoma) cells following treatment with a rhamnan sulfate cell fraction.

FIG. 5 illustrates the general structure of the synthetic pentasaccharide Factor Xa inhibitor fondaparinux.

FIG. 6 illustrates the effect of the RS #1 isoform on LDL permeability of control and tnf/chx-treated HCAEC monolayers. Mean+/−SEM shown. *p<0.05.

FIG. 7 illustrates the effect of the RS #2 isoform on the LDL permeability of control and tnf/chx-treated HCAEC monolayers.

FIG. 8 illustrates the effect of the RS #1 (n=3) and RS #2 (n=2) isoforms on water flux; in this graph, n=4 for control.

FIG. 9A illustrates a representative photomicrograph of heparin sulfate (HS) staining for the control of Dil-LDL bound to HCAEC monolayers after the LDL transport experiment.

FIG. 9B illustrates the fluorescent microscope photomicrograph of HS staining for the RS #1 isoform treated monolayers, showing Dil-LDL bound to HCAEC monolayers after the LDL transport experiment.

FIG. 10 illustrates HS stimulus results of the various RS isoforms.

FIG. 11 illustrates the exemplary ECM binding of the rhamnan sulfate isoforms.

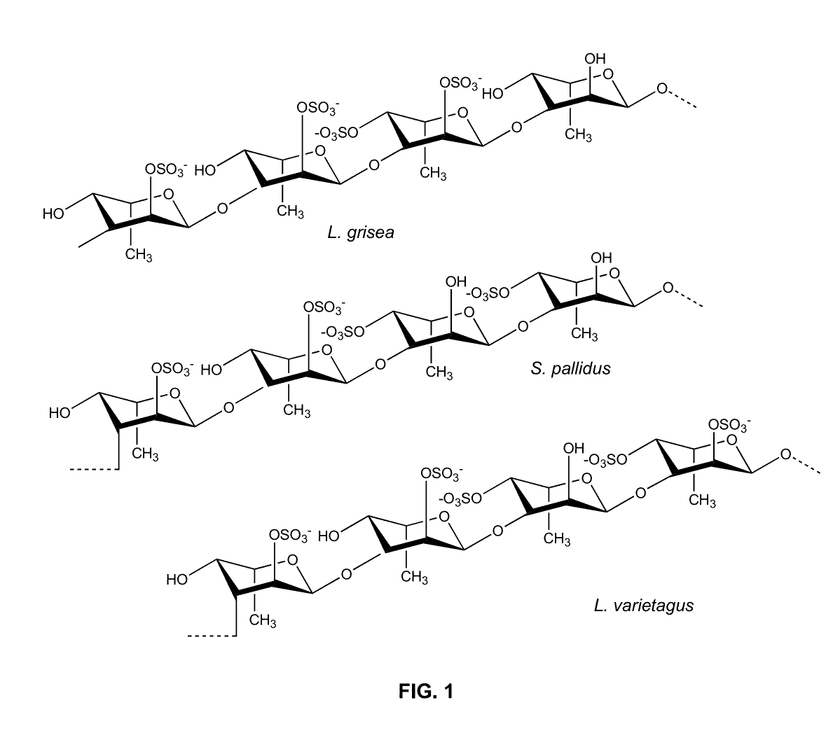

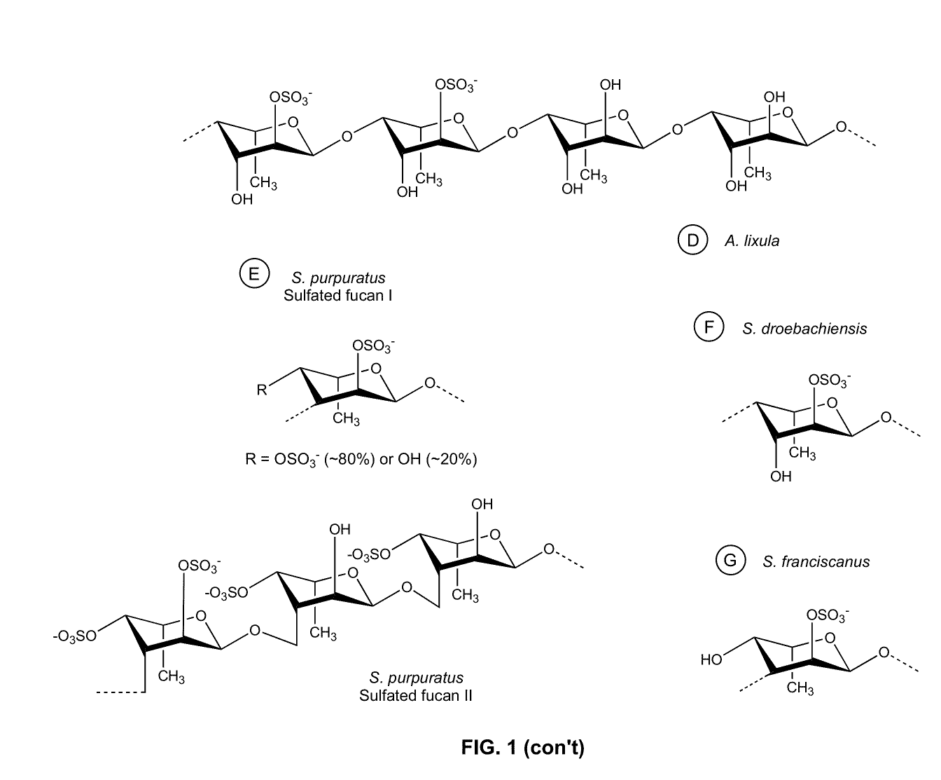

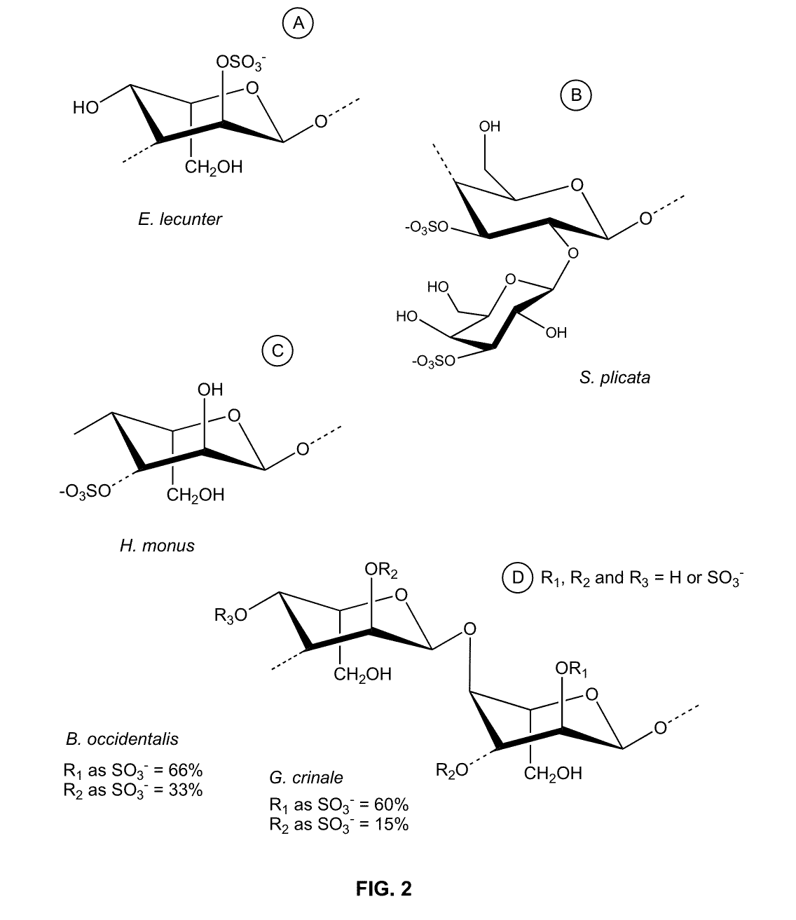

While the inventions disclosed herein are susceptible to various modifications and alternative forms, only a few specific embodiments have been shown by way of example in the drawings and are described in detail below. The figures and detailed descriptions of these specific embodiments are not intended to limit the breadth or scope of the inventive concepts or the appended claims in any manner. Rather, the figures and detailed written descriptions are provided to illustrate the inventive concepts to a person of ordinary skill in the art and to enable such person to make and use the inventive concepts.

### DEFINITIONS

The following definitions are provided in order to aid those skilled in the art in understanding the detailed description of the present invention.

It must be noted that, as used in this specification and the appended claims, the singular forms “a”, “an” and “the” include plural referents unless the content clearly dictates otherwise. Thus, for example, reference to “a NASP” includes a mixture of two or more such agents, and the like.

The term “alkyl”, alone or in combination, as used herein means a straight, branched, or cyclic, primary, secondary, or tertiary saturated hydrocarbon, including those containing from 1 to 10 carbon atoms or from 1 to 6 carbon atoms and can be optionally substituted as described herein for “aryl”. The term alkyl includes fluorinated alkyl such as trifluoromethyl and difluoromethyl.

The term “alkenyl”, alone or in combination, means an acyclic, straight, branched, or cyclic, primary, secondary, or tertiary hydrocarbon, including those containing from 2 to 10 carbon atoms or from 2 to 6 carbon atoms, wherein the substituent contains at least one carbon-carbon double bond. These alkenyl radicals may be optionally substituted as desired for example, with groups as described above for alkyl substituents.

The term “alkynyl” means an unsaturated, acyclic hydrocarbon radical, linear or branched, in so much as it contains one or more triple bonds, including such radicals containing about 2 to 10 carbon atoms or having from 2 to 6 carbon atoms. The alkynyl radicals may be optionally substituted as desired, for example with any of the groups described above for alkyl substitution. Examples of suitable alkynyl radicals include but are not limited to ethynyl, propynyl, hydroxypropynyl, butyn-1-yl, butyn-2-yl, pentyn-1-yl, pentyn-2-yl, 4-methoxypentyn-2-yl, 3-methylbutyn-1-yl, hexyn-1-yl, hexyn-2-yl, hexyn-3-yl, 3,3-dimethylbutyn-1-yl radicals and the like.

The term “acyl”, alone or in combination, means a carbonyl or thionocarbonyl group bonded to any radical to complete the valency, for example selected from, hydrido, alkyl, alkenyl, alkynyl, haloalkyl, alkoxy, alkoxyalkyl, haloalkoxy, aryl, heterocyclyl, heteroaryl, alkylsulfinylalkyl, alkylsulfonylalkyl, aralkyl, cycloalkyl, cycloalkylalkyl, cycloalkenyl, alkylthio, arylthio, amino, alkylamino, dialkylamino, aralkoxy, arylthio, and alkylthioalkyl. Examples of “acyl” functionality groups are formyl, acetyl, benzoyl, trifluoroacetyl, phthaloyl, malonyl, nicotinyl, and the like.

The terms “alkoxy” and “alkoxyalkyl” includes linear or branched oxy-containing radicals each having alkyl portions of, for example, from one to about ten carbon atoms, including the methoxy, ethoxy, propoxy, and butoxy radicals. The term “alkoxyalkyl” also embraces alkyl radicals having one or more alkoxy radicals attached to the alkyl radical, that is, to form monoalkoxyalkyl and dialkoxyalkyl radicals. Other alkoxy radicals are “lower alkoxy” radicals having one to six carbon atoms. Examples of such radicals include methoxy, ethoxy, propoxy, butoxy and tert-butoxy alkyls. The “alkoxy” radicals may be further substituted with one or more halo atoms, such as fluoro, chloro or bromo, to provide “haloalkoxy” radicals. Examples of such radicals include fluoromethoxy, chloromethoxy, trifluoromethoxy, difluoromethoxy, trifluoroethoxy, fluoroethoxy, tetrafluoroethoxy, pentafluoroethoxy, and fluoropropoxy.

The term “alkylamino” includes “monoalkylamino” and “dialkylamino” radicals containing one or two alkyl radicals, respectively, attached to an amino radical. The terms “arylamino” denotes “monoarylamino” and “diarylamino” containing one or two aryl radicals, respectively, attached to an amino radical. The term “aralkylamino”, embraces aralkyl radicals attached to an amino radical, and denotes “monoaralkylamino” and “diaralkylamino” containing one or two aralkyl radicals, respectively, attached to an amino radical. The term aralkylamino further includes “monoaralkyl monoalkylamino” containing one aralkyl radical and one alkyl radical attached to an amino radical.

The term “alkoxyalkyl” is defined as an alkyl group wherein a hydrogen has been replaced by an alkoxy group. The term “(alkylthio)alkyl” is defined similarly as alkoxyalkyl, except a sulfur atom, rather than an oxygen atom, is present.

The term “alkylthio” and “arylthio” are defined as —SR, wherein R is alkyl or aryl, respectively.

The term “alkylsulfonyl” is defined as R—SO₂—, wherein R is alkyl.

As used herein, the term “amine” describes a —NR′R″ group where each of R′ and R″ is independently hydrogen, alkyl, cycloalkyl, heteroalicyclic, aryl or heteroaryl, as these terms are defined herein.

The term “aryl” as used herein refers to a carbocyclic aromatic system containing one, two or three rings wherein such rings may be attached together in a pendent manner or may be fused. Examples of aryl groups include phenyl, benzyl, naphthyl, and biphenyl. The “aryl” group can be optionally substituted where desired, for example, with one or more independently selected from the following groups: of hydroxyl, thiol, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyoxyalkylene, polyol alkyl, alkylcarbonylalkyl, lower alkyl S(O)-lower alkyl, lower alkyl-S(O)₂-lower alkyl, aralkyl lower thioalkyl, heteroaralkyl lower thioalkyl, heterocyclealkyl lower thioalkyl, heteroaryl lower alkyl, heterocycle lower alkyl, heteroarylthio lower alkyl, arylthio lower alkyl, heterocyclethio lower alkyl, heteroarylamino lower alkyl, heterocycleamino lower alkyl, arylsulfinyl lower alkyl, and arylsulfonyl lower alkyl, oxo, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy, heteroarylalkoxy, heterocycleoxy, heterocyclealkoxy, heteroaryl lower alkoxy, heterocycle lower alkoxy, alkylthio, haloalkylthio, thioether, amino, alkylamino, dialkylamino, alkylsulfonylamino, acylamino, arylamino, heteroarylamino, heterocycleamino, oxyalkylamino, amido, imide, sulfonylimide, carboxamido, sulfonamido, amino acid, amino acid esters, amino acid amides, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, hydroxamic acid, tetrazolyl, carbohydrate, or alditol, all of which can be further substituted with one of the same substituents as set out above and can be either unprotected, or protected as necessary, as known to those skilled in the art. In addition, adjacent groups on an “aryl” ring may combine to form a 5- to 7-membered saturated or partially unsaturated carbocyclic, aryl, heteroaryl or heterocyclic ring, which in turn may be substituted as above.

The term “carboxyl”, as used herein, refers to a —C(═O)—O—R′ group, where R′ is as defined herein. When R′ is hydrogen, the carboxyl group is referred to as a carboxylic acid, and when R′ is an alkyl, the carboxyl group is referred to as an ester.

The term “amide” as used herein describes a —NR′—C(═O)— group, a —NR'—C(═O)—R″ group, or a —C(═O)—NR′R″ group, wherein R′ is as defined herein and R″ is as defined herein for R′. An amide is used herein interchangeably with peptide bond in accordance with the present disclosure.

The term “carbocycle”, alone or in combination, as used herein means any stable 3- to 7-membered monocyclic or bicyclic or 7- to 14-membered bicyclic or tricyclic or an up to 26-membered polycyclic carbon ring, any of which may be saturated, partially unsaturated, or aromatic. Examples of such carbocyles include, but are not limited to, cyclopropyl, cyclopentyl, cyclohexyl, phenyl, biphenyl, naphthyl, indanyl, adamantyl, or tetrahydronaphthyl (tetralin).

The term “cation” as used herein refers to any positively charged ion, including the hydrogen ion, H. In one embodiment, the cation is a metal cation, and includes Na⁺, K⁺, Li⁺, Cs⁺, NH₄⁺, as well as transition metal cations.

The term “peptide bond” as used herein refers to an amide group, namely, a —(C═O)NH— group, which is typically formed by a nucleophilic addition-elimination type of chemical reaction between a carboxylic group and an amine group, as these terms are defined herein.

The term “halo” or “halogen”, as used herein, includes independently fluoro (F), bromo (Br), chloro (Cl), and iodo (I) groups.

The term “heterocyclic” and “heterocycle” alone or in combination includes nonaromatic cyclic groups that may be partially (e.g., contains at least one double bond) or fully saturated and wherein there is at least one heteroatom, such as oxygen, sulfur, nitrogen, or phosphorus in the ring. Similarly, the term heteroaryl or heteroaromatic, as used herein, refers to an aromatic ring that includes at least one sulfur, oxygen, nitrogen or phosphorus in the aromatic ring. Nonlimiting examples of heterocylics and heteroaromatics include pyrrolidinyl, tetrahydrofuryl, piperazinyl, piperidinyl, morpholino, thiomorpholino, tetrahydropyranyl, imidazolyl, pyrrolyl, pyrazolyl, indolyl, dioxolanyl, or 1,4-dioxanyl, aziridinyl, furyl, furanyl, chromenyl, chromenyl-4-one, pyridyl, pyrimidinyl, benzoxazolyl, 1,2,4-oxadiazolyl, 1,3,4-oxadiazolyl, 1,3,4-thiadiazole, indazolyl, 1,3,5-triazinyl, thienyl, isothiazolyl, imidazolyl, tetrazolyl, pyrazinyl, benzofuranyl, quinolinyl, isoquinolinyl, benzothienyl, isobenzofuryl, pyrazolyl, indolyl, isoindolyl, benzimidazolyl, purinyl, tetrazolyl, carbazolyl, oxazolyl, thiazolyl, benzothiazolyl, isothiazolyl, 1,2,4-thiadiazolyl, 1,2,3-thiadiazolyl, isoxazolyl, pyrrolyl, quinazolinyl, cinnolinyl, phthalazinyl, xanthinyl, hypoxanthinyl, pyrazole, imidazole, 1,2,3-triazole, 1,2,4-triazole, 1,2,3-oxadiazole, thiazine, pyridazine, or pteridinyl wherein the heteroaryl or heterocyclic group can be optionally substituted with one or more substituents, for example, one of the same substituents as set out above for aryl groups. In addition, adjacent groups on the heteroaryl or heterocyclic ring may combine to form a 5- to 7-membered carbocyclic, aryl, heteroaryl or heterocyclic ring, which in turn may be substituted as above. Functional oxygen and nitrogen groups on the heteroaryl group can be protected as necessary or as desired. Suitable protecting groups can include but are not limited to trimethylsilyl (TMS), dimethylhexylsilyl (DMHS), t-butyldimethylsilyl (TBS or TBDMS), and t-butyldiphenylsilyl (TBDPS), trityl (Trt) or substituted trityl, alkyl groups, acyl (Ac) groups such as acetyl and propionyl, methanesulfonyl, and p-toluenelsulfonyl.

The term “hydrocarbon” as used herein means a group, radical, or compound containing essentially of carbon and hydrogen, and optionally of oxygen, nitrogen, sulfur, or phosphorous atoms, but excluding (not comprising) silicon or fluorine atoms. The term hydrocarbon as used herein includes linear, branched, or cyclic alkyl, alkenyl, alkynyl groups which may be optionally substituted, as well as aryl groups include those with a carbocyclic aromatic system containing one, two or three rings wherein such rings may be attached together in a pendent manner or may be fused. Preferably, the term “hydrocarbon” denotes a group, radical, or compound composed solely of carbon, hydrogen, and optionally oxygen atoms.

The term “branched” as used herein refers to a compound comprising at least one branching substituent from the main chain, the branching substituent comprising at least two carbon atoms. More generally, the number of branchings of a molecule corresponds to the number of side groups comprising at least one carbon atom and branched on the main chain of the molecule, the main chain corresponding to the longest carbon chain of the molecule [see, for example, Organic Chemistry, L. G. Wade, Jr., 2nd Edition, Prentice-Hall, Inc., Chapter 3].

The term “sulfate group” as used herein refers to groups having the general formula —OSO₃R, wherein R is selected from the group consisting of atoms and/or molecules that form monovalent cations, such as H, Na, K, L₁ and NH₄.

The term “sulfonamide” includes both R—SO₂—N—, and R—N—SO₂—, wherein R is aryl, heteraryl, heterocyclic or alkyl.

The terms “protecting group” or “protected” refers to a substituent that protects various sensitive or reactive groups present, so as to prevent said groups from interfering with a reaction. Such protection may be carried out in a number of well-known manners as taught by Greene, et al., “Protective Groups in Organic Synthesis,” John Wiley and Sons, Third Edition, 1999 or the like, depending upon the functional group to be protected. The protecting group may be removed after the reaction in any manner known by those skilled in the art. Non-limiting examples of protecting groups suitable for use within the present invention include but are not limited to allyl, benzyl (Bn), tertiary-butyl (t-Bu), methoxymethyl (MOM), p-methoxybenzyl (PMB), trimethylsilyl (TMS), dimethylhexylsily (TDS)I, t-butyldimethylsilyl (TBS or TBDMS), and t-butyldiphenylsilyl (TBDPS), tetrahydropyranyl (THP), trityl (Trt) or substituted trityl, alkyl groups, acyl groups such as acetyl (Ac) and propionyl, methanesulfonyl (Ms), and p-toluenesulfonyl (Ts). Such protecting groups can form, for example in the instances of protecting hydroxyl groups on a molecule: ethers such as methyl ethers, substituted methyl ethers, substituted alkyl ethers, benzyl and substituted benzyl ethers, and silyl ethers; and esters such as formate esters, acetate esters, benzoate esters, silyl esters and carbonate esters, as well as sulfonates, and borates.

The term “polysaccharide” as used herein refers to a polymer composed of two or more monosaccharides linked to one another. In many polysaccharides, the basic building block of the polysaccharide is actually a disaccharide unit, which can be repeating or non-repeating, branched or non-branching. Thus, a unit when used with respect to a polysaccharide as described herein refers to a basic building block of a polysaccharide and can include a monomeric building block (monosaccharide) or a dimeric building block (disaccharide). Polysaccharides include but are not limited to heparin-like glycosaminoglycans, chondroitin sulfate, hyaluronic acid and derivatives or analogs thereof, chitin derivatives and analogs thereof, e.g., 6-O-sulfated carboxymethyl chitin, immunogenic polysaccharides isolated from phellinus linteus, PI-88 (a mixture of highly sulfated oligosaccharide derived from the sulfation of phosphomannum which is purified from the high molecular weight core produced by fermentation of the yeast pichia holstii) and its derivatives and analogs, polysaccharide antigens for vaccines, and calcium spirulan (Ca-SP, isolated from blue-green algae, spirulina platensis) and derivatives and analogs thereof.

Further exemplary sulfated polysaccharides suitable for use herein include, but are not limited to all plant and non-mammalian sulfated polysaccharides (SPS), including carragenans; alginates; chitosans; beta-glucans; insulin and derivates and analogs thereof; L-saccharides including fructose, mannose, xylose, and rhamnose, as well as epimers and isomers thereof, particularly those having an effect or role in GAG synthesis (e.g., as a chain initiator); as well as mixtures of SPS's; SPS-polyunsaturated fatty acid (PUFA) conjugates; antioxidant-SPS polymers/antioxidant oligomers; cationic co-polymers; absorption enhances; amphiphilic copolymers of SPS; end-functionalized and “within-chain” functionalized SPS; as well as combinations of two or more of any of the above, and pharmaceutically acceptable salts, solvates, hydrates, and prodrugs thereof.

The term “polysaccharide,” as used herein, may also, and equivalently, refer to a polymer comprising a plurality (i.e., two or more) of covalently linked saccharide residues. Linkages may be natural or unnatural. Natural linkages include, for example, glycosidic bonds, while unnatural linkages may include, for example, ester, amide, or oxime linking moieties. Polysaccharides in accordance with the present disclosure may have any of a wide range of average molecular weight (MW) values, but generally are of at least about 100 daltons. For example, the polysaccharides can have molecular weights of at least about 500, 1000, 2000, 4000, 6000, 8000, 10,000, 20,000, 30,000, 50,000, 100,000, 500,000 daltons or even higher. Such polysaccharides may have straight chain or branched structures, and may include fragments of polysaccharides generated by degradation (e.g., hydrolysis) of larger polysaccharides. Degradation can be achieved by any of a variety of means known to those skilled in the art including treatment of polysaccharides with acid, base, heat, or enzymes to yield degraded polysaccharides. Polysaccharides may be chemically altered and may have modifications, including but not limited to, sulfation, polysulfation, esterification, and methylation.

A polysaccharide according to the invention can be a mixed population of polysaccharides, e.g., a heparin, synthetic heparin, or LMWH preparation. As used herein, a “mixed population of polysaccharides” is a polydisperse mixture of polysaccharides. The term “polydisperse” or “polydispersity” refers to the weight average molecular weight of a composition (M_{w}) divided by the number average molecular weight (Mₙ). The polydispersity of unfractionated heparin and various LMWHs are known, as are methods for determining polydispersity. Compositions with polydispersity near 1 are more homogeneous, containing fewer different polysaccharides. As an example, a preparation of unfractionated heparin, which contains a wide variety of polysaccharides of differing lengths and compositions, has a polydispersity of about 1.5 to 2.0.

A polysaccharide in accordance with the present invention may also include polysaccharide extracts, homogenates, and polymer extracts of polysaccharides, in addition to purified, single-entity polysaccharides and polysaccharide polymers. As used herein, the term “extract” means the active ingredients isolated from a natural polysaccharide, and is also intended to encompass salts, complexes, and other derivatives of the extract which possess the herein-described biological characteristics and/or therapeutic indications. The term “extract” is also intended to cover synthetically or biologically produced polysaccharide analogs and homologs with the same or similar characteristics yielding the same or similar biological or therapeutic results as described in the present disclosure.

An “SP” as used herein refers to a ‘sulfated polysaccharide’ that exhibits biological activity in a specific, biologic assay that is no more than one-third, and preferably less than one-tenth, the molar anticoagulant (statistically significant increase in clotting time) activity of unfractionated heparin (MW range 8,000 to 30,000; mean 18,000 daltons). Sulfated polysaccharide's (SPs) suitable for use with the present invention may be purified and/or modified from natural sources (e.g. brown algae, green algae, tree bark, bacteria), excluding animal sources, or may be synthesized de novo and may range in molecular weight from about 100 daltons to about 1,000,000 daltons, inclusive. SPs may be used in the methods of the invention for the treatment of a variety of disorders in patients, particularly those disorders associated with a disruption of, or the destruction of, the glycocalyx. The ability of the SPs of various molecular weights (MWs) and chain lengths to be incorporated into, and diffuse within the extracellular matrix and

Various MWs and chain lengths incorporate into and diffuse within the extracellular matrix and glycocalyx of endothelial and other cells leads to broad effects and applications due to their protective and synergistic effects on the endogenous molecules of the regulatory glycocalyx structure and function. In accordance with the present disclosure, the administered SPS can increase the density, charge, and homeostatic roles of the glycocalyx upon incorporation as they function like the endogenous PS due to their structure and charge. According to aspects of the present disclosure, administered SPS interact synergistically with endogenous PS for promotion of normal homeostatic control of cellular processes. SPS incorporation into the cellular glycocalyx can decrease the permeability for macromolecules, reduce inflammatory processes, promote vasodilation, and control cell-cell orientation, and cell signaling and transcriptional processes.

For example, by their incorporation into the endogenous glycocalyx, the SPS compositions of the present disclosure can decrease endogenous glycocalyx PS and protein breakdown from free radical processes, inhibit glycocalyx degradation from thrombin, heparanase, hyaluronidase, protease, collagenase, elastase, plasmin, chemokines, and other ECM degrading enzymes and processes as the effector molecules breaking down the glycocalyx interact with the charged sulfate and other constituent of the administered SPS thereby limiting breakdown of the analogous PS and proteins native to the ECM-glycocalyx, which in turn could inhibit hyaluronan degradation and inflammation in a patient.

Other advantages of the effect of the SPs compositions of the present disclosure, particularly with respect to the effect on the glycocalyx of a subject, include, but are not limited to, an increase in vivo glycosaminoglycan, heparin, and heparan synthesis for homeostasis, anti-inflammatory, anti-adhesiveness, and anti-thrombotic activities; limiting endothelial dysfunction from free radical, aging, and other processes that lead to pathologic glycocalyx degradation and ineffective endogenous glycocalyx repair; increase nitric oxide (NO) levels for homeostasis promoting, vasodilating, anti-inflammatory, anti-adhesive, and anti-thrombotic activities; increase binding and activity of superoxide dismutase, catalase, and other anti-oxidant regulating molecules within the glycocalyx; increase the antioxidant potential of the glycocalyx through properties related to sulfation and other polysaccharide properties; localization of administered SPS in the glycocalyx may increase endogenous anti-thrombotic and thrombolytic potential in the vasculature without plasma anticoagulant thereby inhibiting vascular occlusion and cellular ischemic injury; increase resistance of cells and tissues to hypoxia, reperfusion injury, oxidative stress, aging and other injurious metabolic processes recognized to cause glycocalyx degradation; decrease the exposure of selectins resulting from endothelial and vascular glycocalyx degradation thereby decreasing binding of WBCs, macrophages, and platelets associated with cellular injury and inflammatory processes; enhance endothelial progenitor cell release, differentiation, and repair through release of EPCs from bone marrow and stem cell differentiation effects; and, exhibit a decreased rate of endothelial cell senescence and apoptosis and thrombogenic endothelial microparticle release.

The therapeutic actions of the SPs compositions of the present disclosure can be readily determined using various assays and models (e.g. histological, microscopic, or other assays in an appropriate animal model). Non-limiting examples of such assays, models, and tests include the use of numerous plasma markers reflecting glycocalyx degradation, inflammation, tissue and endothelial damage, natural anticoagulation, and fibrinolysis may be assayed that reflect the effects of SPS on the structure and function of the glycocalyx including: syndecan-1, perlecan, glypican, IL-6, IL-10, histone-complexed DNA fragments, high-mobility group box 1 (HMGB1), thrombomodulin, von Willebrand factor, intercellular adhesion molecule-1, E-selectin, protein C, tissue factor pathway inhibitor (TFPI), antithrombin, D-dimer, tissue-type plasminogen activator (tPA), urokinase-type plasminogen activator (uPA), soluble uPA receptor, and plasminogen activator inhibitor-1. Additionally, the activity and inhibition of glycocalyx degradation enzymes hyaluronidase, heparanase, elastase, collagenase, metalloproteases, and others may be assessed, as appropriate; assays of nitric oxide production and bioactivity, anti-oxidant capacity, iso-prostane production, vasodilator capacity, and tissue anti-oxidant capacity reflecting effects of SPS on endogenous homeostasis systems may be measured; the improvement or normalization of endothelial function may be reflected in SPS effects on the glycocalyx by numerous mechanical methods including catheter-based methods, venous occlusion plethysmography, high-frequency ultrasound, peripheral arterial tonometry, digital pulse amplitude tonometry, digital thermal monitoring, the L-arginine test and measurement of compounds released by endothelial cells; the identification of endothelial damage and repair reflected in assays demonstrating reduction in numbers of circulating endothelial cells and microparticles may be performed; direct in vivo and in vitro imaging techniques measuring glycocayx dimension and density may be used to demonstrate effects of SPS on glycocalyx structure and function as a permeability barrier for macromolecules; and, the measurement of endothelial progenitor cell release and function from SPS effects can be performed.

The term “derived from” is used herein to identify the original source of a molecule but is not meant to limit the method by which the molecule is made which can be, for example, by chemical synthesis or recombinant means.

The terms “variant,” “analog” and “mutein” refer to biologically active derivatives of the reference molecule, that retain desired therapeutic activity, such as targeting the glycocalyx in the treatment of sickle cell anemia disorders described herein. In general, the terms “variant” and “analog” in reference to a polypeptide (e.g., clotting factor) refer to compounds having a native polypeptide sequence and structure with one or more amino acid additions, substitutions (generally conservative in nature) and/or deletions, relative to the native molecule, so long as the modifications do not destroy biological activity and which are “substantially homologous” to the reference molecule as defined below. In general, the amino acid sequences of such analogs will have a high degree of sequence homology to the reference sequence, e.g., amino acid sequence homology of more than 50%, generally more than 60%-70%, even more particularly 80%-85% or more, such as at least 90%-95% or more, when the two sequences are aligned. Often, the analogs will include the same number of amino acids but will include substitutions, as explained herein. The term “mutein” further includes polypeptides having one or more amino acid-like molecules including but not limited to compounds comprising only amino and/or imino molecules, polypeptides containing one or more analogs of an amino acid (including, for example, unnatural amino acids, etc.), polypeptides with substituted linkages, as well as other modifications known in the art, both naturally occurring and non-naturally occurring (e.g., synthetic), cyclized, branched molecules and the like. The term also includes molecules comprising one or more N-substituted glycine residues (a “peptoid”) and other synthetic amino acids or peptides. [See, e.g., U.S. Pat. Nos. 5,831,005; 5,877,278; and 5,977,301; Nguyen, et al., Chem. Biol. (2000) 7:463-473; and Simon, et al., Proc. Natl. Acad. Sci. USA (1992) 89:9367-9371 for descriptions of peptoids]. Preferably, the analog or mutein has at least the same clotting activity as the native molecule. Methods for making polypeptide analogs and muteins are known in the art.

As explained above, analogs generally include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the polypeptide of interest may include up to about 5-10 conservative or non-conservative amino acid substitutions, or even up to about 15-25 conservative or non-conservative amino acid substitutions, or any integer between 5-25, so long as the desired function of the molecule remains intact. One of skill in the art may readily determine regions of the molecule of interest that can tolerate change by reference to Hopp/Woods and Kyte-Doolittle plots, well known in the art.

As used herein, the term “derivative” is intended to refer to any suitable modification of the reference molecule of interest (specifically an SP) or of an analog thereof, such as sulfation, acetylation, glycosylation, phosphorylation, polymer conjugation (such as with polyethylene glycol), or other addition of foreign moieties, so long as the desired biological activity (e.g., clotting activity, inhibition of TFPI activity) of the reference molecule is retained. For example, polysaccharides may be derivatized with one or more organic or inorganic groups. Examples include polysaccharides substituted in at least one hydroxyl group with another moiety (e.g., a sulfate, carboxyl, phosphate, amino, nitrile, halo, silyl, amido, acyl, aliphatic, aromatic, or a saccharide group), or where a ring oxygen has been replaced by sulfur, nitrogen, a methylene group, etc. Polysaccharides may be chemically altered, for example, to improve procoagulant function. Such modifications may include, but are not limited to, sulfation, polysulfation, esterification, and methylation. Methods for making analogs and derivatives are generally available in the art.

By “fragment” is intended a molecule consisting of only a part of the intact full-length sequence and structure. A fragment of a polysaccharide may be generated by degradation (e.g., hydrolysis) of a larger polysaccharide. Active fragments of a polysaccharide will generally include at least about 2-20 saccharide units of the full-length polysaccharide, preferably at least about 5-10 saccharide units of the full-length molecule, or any integer between 2 saccharide units and the full-length molecule, provided that the fragment in question retains biological activity, such as clotting activity and/or the ability to provide a generally therapeutic benefit to a subject to which such a molecule is administered. A fragment of a polypeptide can include a C-terminal deletion an N-terminal deletion, and/or an internal deletion of the native polypeptide. Active fragments of a particular protein will generally include at least about 5-10 contiguous amino acid residues of the full-length molecule, preferably at least about 15-25 contiguous amino acid residues of the full-length molecule, and most preferably at least about 20-50 or more contiguous amino acid residues of the fill-length molecule, or any integer between 5 amino acids and the full-length sequence, provided that the fragment in question retains biological activity, such as clotting activity, as defined herein.

“Substantially purified” as used herein generally refers to isolation of a substance (e.g., sulfated polysaccharide) such that the substance comprises the majority percent of the sample in which it resides. Typically in a sample a substantially purified component comprises 50%, preferably 80%-85%, more preferably 90-95% of the sample. Techniques for purifying sulfated polysaccharides of interest are well-known in the art and include, for example, ion-exchange chromatography, affinity chromatography and sedimentation according to density.

The term “isolated” as used herein is meant, when referring to a polysaccharide or polypeptide, that the indicated molecule is separate and discrete from the whole organism with which the molecule is found in nature or is present in the substantial absence of other biological macro-molecules of the same type.

“Homology” refers to the percent identity between two polynucleotide or two polypeptide moieties. Two nucleic acid, or two polypeptide sequences are “substantially homologous” to each other when the sequences exhibit at least about 50%, preferably at least about 75%, more preferably at least about 80%-85%, preferably at least about 90%, and most preferably at least about 95%-98% sequence identity over a defined length of the molecules. As used herein, substantially homologous also refers to sequences showing complete identity to the specified sequence.

In general, “identity” refers to an exact nucleotide-to-nucleotide or amino acid-to-amino acid correspondence of two polynucleotides or polypeptide sequences, respectively, although it may also refer equally to an SP-to-SP structural correspondence, particularly between a synthetic and an isolated SP. Percent identity can be determined by a direct comparison of the sequence or structural information between two molecules (the reference sequence and a sequence with unknown % identity to the reference sequence) by aligning the sequences, counting the exact number of matches between the two aligned sequences, dividing by the length of the reference sequence, and multiplying the result by 100. Readily available computer programs can be used to aid in the analysis, such as ALIGN; Dayhoff, M. O. in Atlas of Protein Sequence and Structure M. O. Dayhoff ed., 5 Suppl. 3:353-358, National biomedical Research Foundation, Washington, D.C., which adapts the local homology algorithm of Smith and Waterman Advances in Appl. Math. 2:482-489, 1981 for peptide analysis. Programs for determining nucleotide sequence identity are available in the Wisconsin. Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters; details of these programs are readily available.

The term “recombinant” as used herein to describe a nucleic acid molecule means a polynucleotide of genomic, cDNA, viral, semisynthetic, or synthetic origin which, by virtue of its origin or manipulation is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

By “vertebrate subject” is meant any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The invention described herein is intended for use in any of the above vertebrate species.

The term “patient,” as used herein, refers to a living organism suffering from or prone to a condition that can be prevented or treated by administration of a SP of the invention, and includes both humans and animals.

As used herein, a “biological sample” refers to a sample of tissue or fluid isolated from a subject, including but not limited to, for example, blood, plasma, serum, fecal matter, urine, bone marrow, bile, spinal fluid, lymph fluid, samples of the skin, external secretions of the skin, respiratory, intestinal, and genitourinary tracts, tears, saliva, milk, blood cells, organs, biopsies and also samples of in vitro cell culture constituents including but not limited to conditioned media resulting from the growth of cells and tissues in culture medium, e.g., recombinant cells, and cell components.

As used herein, the term “therapeutically effective dose or amount” of a SP, or other therapeutic agent is intended to refer to an amount that, when administered as described herein, brings about a positive therapeutic response, such as reduced bleeding or shorter clotting times.

The term “treating” or “treatment” of any disease or disorder refers, in some embodiments, to ameliorating the disease or disorder (i.e., arresting or reducing the development of the disease or at least one of the clinical symptoms thereof). In other embodiments “treating” or “treatment” refers to ameliorating at least one physical parameter, which may not be discernible by the patient. In yet other embodiments, “treating” or “treatment” refers to inhibiting the disease or disorder, either physically, (e.g., stabilization of a discernible symptom), physiologically, (e.g., stabilization of a physical parameter) or both. In yet other embodiments, “treating” or “treatment” refers to delaying the onset of the disease or disorder. “Treating” or “treatment” as used herein covers the treatment of the disease or condition of interest, e.g., vascular tissue injury, in a mammal, preferably a human, having the disease or condition of interest, and includes: (i) preventing the disease or condition from occurring in a mammal, in particular, when such mammal is predisposed to the condition but has not yet been diagnosed as having it; (ii) inhibiting the disease or condition, i.e., arresting its development; (iii) relieving the disease or condition, i.e., causing regression of the disease or condition; or (iv) relieving the symptoms resulting from the disease or condition.

As used herein, the terms “disease,” “disorder,” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

As used herein, the term “vitamin” refers to those compounds which are considered to be nutrients required for essential metabolic reactions within the body, and which are capable of acting both as catalysts and participants in chemical reactions within the body of mammals [Kutsky, R. J. Handbook of Vitamins and Hormones. Van Nostrand Reinhold, New York (1973); Kirk-Othmer, Encyclopedia of Chemical Technology, Third Edition, John Wiley and Sons, NY, Vol. 24:104 (1984)].

The term “mortality” as used herein relates to mortality from any cause, preferably, from a cardiovascular complication or cardiovascular event. The term “cardiovascular event” as used herein refers to any disorder of the cardiovascular system including preferably any acute cardiovascular event. Acute cardiovascular events are, preferably, stable angina pectoris (SAP) or acute coronary syndrome (ACS). ACS patients can show unstable angina pectoris (UAP) or myocardial infarction (MI). MI can be an ST-elevation MI (STEM) or a non-ST-elevation MI (NSTEMI). NSTE-ACS as used herein encompasses UAP and NSTEMI. The occurrence of an MI can be followed by a left ventricular dysfunction (LVD), development of heart failure or even mortality. Further preferred cardiovascular events encompass cardiac brady- or tachyarrhythmias including sudden cardiac death and stroke (cerebrovascular events or accidents). Also, mortality can refer to the death rate or the ratio of number of deaths to a given population of subjects.

The terms “modulate” or “modulating”, as used in the instant application, refer to both therapeutic treatment and prophylactic or preventative measures, wherein the object is to prevent or slow down (lessen) an undesired clinical parameter. For purposes of the present invention, beneficial or desired clinical results include, but are not limited to, correcting of clinical parameter, diminishment of extent of clinical parameter, stabilized (i.e., not worsening) clinical parameter and delay or slowing of extent of a clinical parameter related to a disease or disorder treated in accordance with the present invention, using the therapeutic compositions as described in the instant application.

The present embodiments further encompass any enantiomers, prodrugs, solvates, hydrates and/or pharmaceutically acceptable salts of the polymers described herein.

As used herein, the term “enantiomer” refers to a stereoisomer of a polymer that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems.

The term “prodrug” refers to an agent, which is converted into the active polymer (the active parent drug) in vivo. Prodrugs are typically useful for facilitating the administration of the parent drug. They may, for instance, be bioavailable by oral administration whereas the parent drug is not. A prodrug may also have improved solubility as compared with the parent drug in pharmaceutical compositions. Prodrugs are also often used to achieve a sustained release of the active compound in vivo. An example, without limitation, of a prodrug would be a compound of the present invention, having one or more carboxylic acid moieties, which is administered as an ester (the “prodrug”). Such a prodrug is hydrolyzed in vivo, to thereby provide the free compound (the parent drug). The selected ester may affect both the solubility characteristics and the hydrolysis rate of the prodrug.

The term “solvate” as used herein refers to a complex of variable stoichiometry (e.g., di-, tri-, tetra-, penta-, hexa-, and so on), which is formed by a solute (the SP compositions of the present disclosure) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

As used herein, the term “%” when used without qualification (as with w/v, v/v, or w/w) means % weight-in-volume for solutions of solids in liquids (w/v), % weight-in-volume for solutions of gases in liquids (w/v), % volume-in-volume for solutions of liquids in liquids (v/v) and weight-in-weight for mixtures of solids and semisolids (w/w), such as described in Remington's Pharmaceutical Sciences [Troy, David B., Ed.; Lippincott, Williams and Wilkins; 21st Edition, (2005)]

The terms “patient” and “subject”, as used herein, are used interchangeably and refer generally to a mammal, and more particularly to human, ape, monkey, rat, pig, dog, rabbit, cat, cow, horse, mouse, sheep and goat. In accordance with this definition, lung surfaces or membranes described and referenced in accordance with this disclosure refer to those of a mammal, preferably a human or an animal test subject, such as a sheep.

The term “drug” as used in conjunction with the present disclosure means any compound which is biologically active, e.g., exhibits or is capable of exhibiting a therapeutic or prophylactic effect in vivo, or a biological effect in vitro. Several in vivo and in vitro methods can be used to measure product quality bioavailability and establish bioequivalence. These include pharmacokinetic, pharmacodynamic, clinical, and in vitro studies.

As used herein, “pharmacokinetic” refers to the kinetics of release of the drug substance from the drug product into the systemic circulation, as well as clearance, volume of distribution, and absorption, as determined by physiological variables (e.g. gastric emptying, motility, pH). Pharmacokinetics may be evaluated in an accessible biological matrix such as blood, plasma, and/or serum. Pharmacokinetic measurements may also include AUC, does-dependency of activity, peak levels in plasma, time to peak, disposition half-life, and terminal half-life.

As used herein, “pharmaeodynamic” refers to defining factors that cause variability in clinical drug response using general assessments, including bone densitometry and caliper total body fat; pulmonary assessments, including pulmonary function testing, expired nitric oxide, pulmonary imaging; Cardiovascular assessments, including cardiac monitoring, ambulatory blood pressure; Holter monitoring, telemetry, ECG, vital signs, cardiac imaging; Nervous system assessments, including electroencephalography, mental function testing, psychomotor function testing, pharmacokinetic EEG; ENT assessments, including audiometric testing, acoustic rhinometry, intraocular pressure, digital retinography; and gastrointestinal assessments, including gastric pH monitoring, endoscopy, imaging, and/or gastric motility.

### DETAILED DESCRIPTION

The written description of specific structures and functions below are not presented to limit the scope of what Applicants have invented or the scope of the appended claims. Rather, the written description is provided to teach any person skilled in the art to make and use the inventions for which patent protection is sought. Those skilled in the art will appreciate that not all features of a commercial embodiment of the inventions are described or shown for the sake of clarity and understanding. Persons of skill in this art will also appreciate that the development of an actual commercial embodiment incorporating aspects of the present inventions will require numerous implementation-specific decisions to achieve the developer's ultimate goal for the commercial embodiment. Such implementation-specific decisions may include, and likely are not limited to, compliance with system-related, business-related, government-related and other constraints, which may vary by specific implementation, location and from time to time. While a developer's efforts might be complex and time-consuming in an absolute sense, such efforts would be, nevertheless, a routine undertaking for those of skill in this art having benefit of this disclosure. It must be understood that the inventions disclosed and taught herein are susceptible to numerous and various modifications and alternative forms. Lastly, the use of a singular term, such as, but not limited to, “a,” is not intended as limiting of the number of items. Also, the use of relational terms, such as, but not limited to, “top,” “bottom,” “left,” “right,” “upper,” “lower,” “down,” “up,” “side,” and the like are used in the written description for clarity in specific reference to the Figures and are not intended to limit the scope of the invention or the appended claims.

Compounds, pharmaceutical compositions, methods and uses for the treatment of a variety of disorders in a subject are described and provided, the disorders including: sickle-cell anemia, suppressing CRP, such as by attaching to the glycocalyx of an individual; enhancing clot resistance in a subject; decreasing the attachment of LDL and/or inflammation cells to a vascular (e.g., arterial) surface; vascular and endothelial tissue diseases and/or disorders, particularly those having the glycocalyx associated therewith; metabolic syndromes; and abnormal cellular proliferation disorders.

It has also been discovered that the use of naturally-occurring sulfated polysaccharides, such as rhamnan sulfate and the like, in therapeutic treatments of cells has a physiologically therapeutic effect on subjects by reinforcing homeostatic pathways, particularly in those diseases and disorders in which heparan or heparan sulfate is administered. While not wishing to be bound by any particular hypotheses, this therapeutic effect is believed to be tied to the electronegative character and flexability of these polymers, which by binding thereto act to induce heparan synthesis in vivo, thereby acting to cause cells to synthesize heparin in vivo and negating the need to administer heparin or heparin sulfate. Thus, the sulfated polysaccharides appear to be preventing diseases and disorders that HS is administered for, as the cells are induced to make their own heparin by the administration of sulfated polysaccharides.

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

A. Disorders.

Glycocalyx-Based Therapy.

As described herein, non-animal derived sulfated polysaccharides of varying chain length interact with the glycocalyx of a variety of cells, including heart cells and endothelial cells, so as to provide a therapeutic effect.

As used herein, the term “glycocalyx” generally refers to a polysaccharide-rich extracellular matrix on the luminal surface of vascular endothelial cells. The

Glycocalyx is primarily comprised of proteoglycans, glycosaminoglycans and glycoproteins (e.g., selectins, adhesion molecules, etc.) which associate in vivo with water and numerous molecules including inter alia plasma proteins, lipids and enzymes from the circulating blood.

The “status of the glycocalyx” refers to the condition of the glycocalyx, particularly within a subject, at a particular point of time including the relative position. The status of the glycocalyx can be characterized by glycocalyx parameters. The term “alteration” with reference to glycocalyx parameters (e.g., volume or dimension, width, permeability, enzyme activity, etc.) generally encompasses any direction (e.g., increase or decrease) and extent of such alteration. For example, a “decrease” in a value of a parameter may include decreases by at least about 10%, or by at least about 20%, or by at least about 30%, or by at least about 40%, or by at least about 50%, or by at least about 60%, or by at least about 70%, or by at least about 80%, or by at least about 90%, compared to a relevant reference value, such as for example the value of said parameter in a control (healthy) subject. For example, an “increase” in a value of a parameter may include increases by at least about 10%, or by at least about 20%, or by at least about 40%, or by at least about 60%, or by at least about 80%, or by at least about 100%, or by at least about 150% or 200% or even by at least about 500% or like, compared to a relevant reference value, such as for example the value of said parameter in a control (healthy) subject. It will be appreciated that alteration of the glycocalyx parameters results in an alteration of the status of the glycocalyx.

More typically, a condition which involves glycocalyx degeneration may be characterized by a decrease in glycocalyx volume or dimension, increased glycocalyx permeability, increased shedding of glycocalyx (i.e., resulting in reduced thickness of the glycocalyx layer), increased activity of glycocalyx-degrading enzyme(s) and/or decreased activity of glycocalyx-synthesising enzyme(s). Desirably, a treatment intervention, such as, for example disclosed herein, would reverse these trends.

The term “enzyme of glycocalyx metabolism” generally encompasses enzymes which participate in the anabolism (i.e., formation) or catabolism (i.e., degradation) of glycocalyx or of one or more of its components. By preference, but without limitation, the term includes hyaluronidase, myeloperoxidase, heparinase and other exo- and endoglycosidases.

As noted, the above characteristics of endothelial glycocalyx may be advantageously determined by means of detecting one or more glycocalyx-related markers in a sample removed from a subject. In particular, such markers may include glycocalyx-derived molecules, glycocalyx-metabolism enzymes and/or endogenous or exogenous substances that can normally associate with glycocalyx.

Glycocalyx-derived molecules particularly suitable for detection herein include, without limitation, hyaluronan, heparan sulphate (HS), dermatan sulphate, syndecan-1 and total plasma glycosaminoglycan (GAG) content. Commonly, such molecules may be released into bloodstream due to ongoing glycocalyx degradation or shedding, and can thus indicate degenerative alterations of glycocalyx.

Glycocalyx-metabolism enzymes particularly suitable for detection or diagnostic applications herein include, without limitation, hyaluronidase, myeloperoxidase, heparinase and other exo- and endoglycosidases. For example, increased or decreased circulating levels of such enzymes may indicate ongoing enzymatic reactions catalysed thereby, allowing a physician to assess the glycocalyx homeostasis.

Endogenous and exogenous glycocalyx-associating substances particularly suitable for detection herein include, without limitation, glycocalyx permeating tracer molecules such as inter alia dextran 40, or endogenous lectin-like proteins which normally associate with glycocalyx. For example, the circulating amount of such substances, optionally following their injection or infusion, provide a representation of the capacity of glycocalyx to deplete said substances from the bloodstream and thereby an estimate of glycocalyx volume or dimension and molecular accessibility.

Exemplary therapeutic effects associated with effects of the SP-containing compositions of the present disclosure include, but are not limited to, endothelial activation and dysfunction roles in the pathophysiology of sickle cell disease (SCD) related vaso-occlusion. The extent of endothelial activation is related to the presence of severe disease related manifestations such as pulmonary hypertension. In the quiescent state, the endothelium is shielded from circulating blood cells and proteins by the glycocalyx, a highly hydrated cell free mesh of membrane-associated proteoglycans, glycosaminoglycans, glycoproteins and glycolipids located at the endothelial surface. With a thickness ranging from 0.5-3.0 μm, it exceeds the intra-luminal size of most endothelial adhesion molecules, thereby preventing interactions of the endothelium with blood constituents. Glycocalyx volume is regulated by numerous factors and rapid degradation has been observed during ischemia, hypoxia, and exposure to both tumor necrosis factor alpha and lipoproteins. Therefore, in conditions of ischemia and inflammation, which occur continuously in SCD, a reduced glycocalyx volume facilitates interactions of activated endothelial cells with blood cells, proteins, lipids, and other blood constituents.

Until recently, it was generally believed that the characteristic sickle shaped erythrocytes clogged the blood vessels and thereby caused the typical SCD related complications. It is now recognized that clogging of blood vessels occurs upon adhesion of leukocytes to the vessel wall cells. Sickle cells are now recognized to become entrapped secondary to leukocyte adhesion processes. Consequently, the paradigm has changed profoundly and at the present endothelial activation and subsequent leukocyte adhesion and secondary trapping of sickle cells resulting in stasis and subsequent ischemia reperfusion damage is the new paradigm in the pathophysiology of SCD. The attachment of leucocytes to the SCD endothelium is the result of exposure on the endothelium of selectin and other receptors exposed as a consequence of endothelial glycocalyx destruction. Destruction of the glycocalyx in SCD reduces the anti-thrombotic and thrombolytic potentials of the endothelium, alters normal nitric oxide function, and creates a chronic state of endothelial activation and inflammation, e.g., there are fluctuating levels. In certain instances, it has been shown that increased levels of the inflammatory biomarker C-reactive protein at baseline are associated with childhood sickle cell vasocclusive crises. The inflammatory mediators, platelet, leucocyte, and endothelial activation, and ischemia/reperfusion are all operative in SCD. Each is recognized to destroy the endothelial glycocalyx through production and release of heparanases, proteases, oxidants, and other molecules that destroy the glycocalyx thereby exacerbating and reinforcing these injurious processes. Antioxidants attenuate blood flow abnormalities in experimental models of SCD and reactive oxygen species and oxidative intermediates recognized to damage the glycocalyx are known to contribute to the vaso-occlusive processes in SCD.

Metabolic Syndromes.

Metabolic syndrome is characterized by a group of metabolic risk factors in one person including abdominal obesity, insulin resistance or glucose intolerance, atherogenic dyslipidemia, prothrombotic state, proinflammatory state and hypertension. The Adult Treatment Panel defines metabolic syndrome as present if a patient manifests at least three of the following symptoms: waist measuring at least 40 inches for men, 35 inches for women; serum triglyceride levels of at least 150 mg/dl; HDL cholesterol levels of less than 40 mg/dl in men, less than 50 mg/dl in women, blood pressure of at least 135/80 mm Hg and blood sugar (serum glucose) of at least 110 mg/dl.

Although there is no complete agreement on the individual risk or prevalence of each factor or symptom (depending upon the definition), it is known that the syndrome poses a significant health risk to individuals. A person having one factor associated with the syndrome has an increased risk for having one or more of the others. The more factors that are present, the greater the risks to the person's health. When the factors are present as a group, i.e., a metabolic syndrome, the risk for cardiovascular disease and premature death is very high. It has been estimated that up to 25% of the population in the United States are afflicted with metabolic syndrome.

An underlying cause of metabolic syndrome is believed to be insulin resistance wherein the ability of insulin to take in glucose from the blood is attenuated. This causes glucose levels to remain elevated after eating to which the pancreas responds by excreting insulin. If left untreated, metabolic syndrome significantly increases the risk of type II diabetes, cardiovascular disease and other diseases related to plaque buildups in artery walls. The term “metabolic syndrome” is well known by the skilled person. As used herein the term, preferably, relates to a cluster of risk factors including hypertriglyceridemia, abdominal obesity, arterial hypertony, and various metabolic disorders including dyslipidaemia and hyperglycemia, as well as diabetes and diabetic retinopathy. In the art, different terms are known for the metabolic syndrome such as metabolic syndrome X, syndrome X, insulin resistance syndrome, and Reaven's syndrome. The, the term “metabolic syndrome” as used herein, relates to metabolic syndromes as defined by the Adult Treatment Panel or any other recognized definition of this syndrome. Synonyms for “metabolic syndrome” used in the art include Reaven's Syndrome, Insulin Resistance Syndrome and Syndrome X. Thus, it is to be understood that where the term “metabolic syndrome” is used herein it also refers to Reaven's Syndrome, Insulin Resistance Syndrome and Syndrome X.

It is also known that various criteria exist for identifying individuals having the metabolic syndrome. Preferably, the metabolic syndrome as used herein is defined by the criteria according to the WHO (World Health Organization, see also Definition, Diagnosis and Classification of Diabetes Mellitus and its Complications; Part 1: Diagnosis and Classification of Diabetes Mellitus. Geneva: WHO Department of Noncommunicable Disease Surveillance; 1999), or by the criteria according to the EGIR (European Group for the Study of Insulin Resistance), or by the criteria according to the NCEP (National Education Program Adult Treatment Panel III, frequently also referred to as ATP III criteria) or by the AHA/NHLBI criteria (American Heart Association/Updated NCEP). Of these the EGIR criteria are more preferred, and the ATP III criteria are most preferred for the diagnosis of a metabolic syndrome. The various criteria defined by the organizations are known in the art (see, for a review Scott M. Grundy et al., Circulation; 2005(112):2735-2752 which hereby is incorporated by reference in its entirety with respect to the disclosure content). In the context of the present invention, a subject who fulfills the specific criteria as defined by the various organizations as referred to herein suffers from the metabolic syndrome, and, thus, has developed a metabolic syndrome.

The criteria according to the WHO (1999) require, for the diagnosis of a metabolic syndrome in a subject, the presence of insulin resistance identified by one of the following: diabetes mellitus, impaired glucose tolerance, impaired fasting glucose or for those with normal fasting glucose levels (<110 mg/dL) glucose uptake below the lowest quartile for background population under investigation under hyperinsulinemic, euglycemic conditions. In addition to insulin resistance, the presence at least two of the following is required: a) blood pressure: ≧140 mmHg systolic or ≧90 mmHg diastolic (or on treatment for high blood pressure); b) plasma triglycerides (TG): 1.695 mmol/L and high-density lipoprotein cholesterol (HDL C) ≦0.9 mmol/L (for male subjects), ≦1.0 mmol/L (for female subjects); c) waist to hip ratio >0.90 (for male subjects); >0.85 (for female subjects), and/or body mass index (BMI)>30 kg/m²; and d) urinary albumin excretion ratio ≧20 μg/min or albumin to creatinine ratio ≧30 mg/g.

The US National Cholesterol Education Program Adult Treatment Panel III [ATP-III/NCEP, 2001, see also: Expert Panel on Detection, Evaluation, and Treatment of High Blood Cholesterol in Adults. Executive Summary of The Third Report of The National Cholesterol Education Program (NCEP) Expert Panel on Detection, Evaluation, And Treatment of High Blood Cholesterol In Adults (Adult Treatment Panel III). JAMA 2001; 285:2486-97, or National Cholesterol Education Program (NCEP) Expert Panel on Detection, Evaluation, and Treatment of High Blood Cholesterol in Adults (Adult Treatment Panel III) Third report of the National Cholesterol Education Program (NCEP) Expert Panel on Detection, Evaluation, and Treatment of High Blood Cholesterol in Adults (Adult Treatment Panel III) final report Circulation 2002; 106:3143-3421] requires, for the presence/diagnosis of a metabolic syndrome, at least three of the following: a) abdominal obesity given as waist circumference ≧102 cm (for male subjects), ≧88 cm (for female subjects); b) plasma triglycerides ≧1.695 mmol/L (150 mg/dl); c) HDL-C ≦40 mg/dL (for male subjects), ≦50 mg/dL (for female subjects); d) blood pressure ≧130/≧85 mmHg; e) fasting plasma glucose ≧6.1 mmol/L (110 mg/dl).

The European Group for the Study of Insulin Resistance (EGIR) requires for the diagnosis/presence of a metabolic syndrome: insulin resistance defined as the top 25% of the fasting insulin values among non-diabetic individuals, and two or more of the following [see also Balkau B, Charles M A, European Group for the Study of Insulin Resistance (EGIR) Comment on the provisional report from the WHO consultation, Diabet Med 1999; 16:442-443]: a) central obesity: waist circumference ≧94 cm (male), ≧80 cm (female) and/or body mass index (BMI) ≦30 kg/m²; b) plasma triglycerides ≧150 mg/dL and/or HDL-C<39 mg/dL (or treated for dyslipidaemia); c) hypertension: blood pressure ≧140/90 mm Hg (or antihypertensive medication); d) fasting plasma glucose ≧6.1 mmol/L.

The AHA/NHLBI criteria (American Heart Association/Updated NCEP, see also Grundy S M, Brewer H B, Cleeman J I, Smith S C, Lenfant D, for the Conference Participants. Definition of metabolic syndrome: report of the National, Heart, Lung, and Blood Institute/American Heart Association conference on scientific issues related to definition. Circulation. 2004; 109:433-438) require, for the presence/diagnosis of a metabolic syndrome, at least three of the following: elevated waist circumference: (Men: Equal to or greater than 102 cm, women equal to or greater than 88 cm); elevated triglycerides (equal to or greater than 150 mg/dl); reduced HDL cholesterol (men: less than 40 mg/dL; women: less than 50 mg/dL); elevated blood pressure (equal to or greater than 130/85 mm Hg or use of medication for hypertension) elevated fasting glucose (equal to or greater than 100 mg/dL (5.6 mmol/L) or use of medication for hyperglycemia).

Generally, patients suffering from the syndrome are prescribed a change in lifestyle, i.e., an increase in exercise and a change to a healthy diet. The goal of exercise and diet programs is to reduce body weight to within 20% of the “ideal” body weight calculated for age and height. In some cases, diet and exercise regimens are supplemented with treatments for lipid abnormalities, clotting disorders, and hypertension. For example, patients with the syndrome typically have several disorders of coagulation that make it easier to form blood clots within blood vessels. These blood clots are often a precipitating factor in developing heart attacks. Patients with the syndrome are often placed on daily aspirin therapy to specifically help prevent such clotting events. Furthermore, high blood pressure is present in more than half the people with the syndrome, and in the setting of insulin resistance, high blood pressure is especially important as a risk factor. Some studies have suggested that successfully treating hypertension in patients with diabetes can reduce the risk of death and heart disease by a substantial amount. Additionally, patients have been treated to specifically reduce LDL-cholesterol (interchangeably referred to herein as “LDL”) levels, reduce triglyceride levels, and raise HDL levels. Drug treatment of metabolic syndrome as a whole has typically included treatment with a statin or a combination of a statin with either niacin or a fibrate in order to focus on the factors individually. Unfortunately, this has met with limited success to date.

The compositions of the present disclosure can be used for the prevention and/or treatment of one or more metabolic syndromes, including insulin resistance syndrome, the treatment methods comprising administering one or more non-animal derived sulfated polysaccharides (or a salt, solvate, hydrate, or prodrug thereof) to a subject in need of such treatment, in an amount being not lower than about 0.0000001% by weight but lower than about 100% by weight, inclusive. Exemplary daily dosage rates for such application include, but are not limited to, 0.1 to 3,000 mg/kg, more preferably 1 to 300 mg/kg. Such compositions may be eaten or drunk (as when formulated as a comestible), and may be applied to human subjects, as well as domestic animals or pets.

Atherosclerotic Disease.

The instant invention further provides, in select embodiments, compositions and methods for preventing or reducing the risk of developing atherosclerosis, as well as for halting or slowing the progression of an atherosclerotic disease once it has become clinically evident. The methods generally comprise administering a therapeutically effective amount of a sulfated polysaccharide, such as rhamnan sulfate and the like, particularly such as sulfated polysaccharide having a non-animal source, alone or in combination with another therapeutic agent and/or other pharmaceutical excipients, to a person who is at risk of developing atherosclerosis, or who already has atherosclerotic disease.

As used herein, the term atherosclerosis will be understood to encompasses vascular diseases and conditions that are recognized and understood by physicians practicing in the relevant fields of medicine. Atherosclerotic cardiovascular disease, coronary heart disease (also known as coronary artery disease or ischemic heart disease), cerebrovascular disease and peripheral vessel disease are all clinical manifestations of atherosclerosis and are therefore encompassed by the terms “atherosclerosis” and “atherosclerotic disease.”

Compositions comprised of one or more sulfated polysaccharides may be administered to prevent or reduce the risk of occurrence, or recurrence where the potential exists, of a coronary heart disease event, a cerebrovascular event, and/or intermittent claudication, in a subject. Coronary heart disease events are intended to include CHD death, myocardial infarction (i.e., a heart attack), and coronary revascularization procedures. Cerebrovascular events are intended to include ischemic or hemorrhagic stroke (also known as cerebrovascular accidents) and transient ischemic attacks. Intermittent claudication is a clinical manifestation of peripheral vessel disease. The term “atherosclerotic disease event” as used herein is intended to encompasses coronary heart disease events, cerebrovascular events, and intermittent claudication. It is also intended that persons who have previously experienced one or more non-fatal atherosclerotic disease event are those for whom the potential for recurrence of such an event exists.

Closely related to this therapeutic application of the compositions described herein are the use of such compositions for the treatment of one or more vascular diseases or disorders. The term “vascular disease or disorder” as used herein refers to any disease or disorder effecting the vascular system, including the heart and blood vessels. A vascular disease or disorder includes any disease or disorder characterized by vascular dysfunction, including, for example, intravascular stenosis (narrowing) or occlusion (blockage), due to the development of atherosclerotic plaque and diseases and disorders resulting therefrom. Examples of vascular diseases and disorders include, without limitation, atherosclerosis, CAD, MI, ischemia, stroke, peripheral vascular diseases, venous thromboembolism and pulmonary embolism. The term vascular disease may also include, hyperproliferative vascular disorders (e.g. restenosis and vascular occlusion), graft vascular atherosclerosis, cardiovascular disease, cerebral vascular disease and peripheral vascular disease (e.g. coronary artery disease, arteriosclerosis, atherosclerosis, nonatheromatous arteriosclerosis or vascular wall damage). It is also used to refer to diseases involving the neogenesis or proliferation of blood vessels in the eye, in particular choroidal neovascularisation

Sickle-Cell Anemia.

Sickle cell anemia is a hereditary blood disease that afflicts certain members of the human race of a variety of ancestries. The anemia results from the physical aggregation of the hemoglobin protein constituent in red blood cells. This aggregation results in a distortion in shape of deoxygenated red blood cells and causes impairment of flow of the blood through the capillaries (sickle cell “crises”). As the principal function of hemoglobin is to transport oxygen from the lungs to body tissues, efficient flow of oxygen throughout the body's tissues is impeded by the anemia due to a lower number of red blood cells. Sickle cell anemia also may have an indirect effect on the heart, lungs, kidneys, spleen, hips and brain. Sickle cell anemia crises can be extremely painful, can result in infections such as pneumonia, can result in skin ulceration, can contribute to strokes and seizures in the one afflicted and can also result in the development of chronic bone infections.

In general, the result of the differences between cells containing hemoglobin A, the normal hemoglobin, and hemoglobin S, the sickle cell hemoglobin, is that the former cell is generally flexible and bioconcave discoid in shape while the latter is more rigid and crescent shaped and typically has pointed ends. This rigidity and distortion in shape cause the cells to be lodged in the capillary. Hemoglobin molecules contain two beta polypeptide chains and two alpha polypeptide chains. In the sickle cell hemoglobin, a mutation is present in the beta chains. More specifically, the sixth amino acid of each beta chain is changed from glutamic acid to valine. As a result of this mutation, hemoglobin S upon deoxygenation polymerizes and causes the cell to assume the elongated, sickle-like configuration. As the sickle cells have a much shorter life span than normal red cells, the effect on the body is to deplete the total volume of blood cells, thereby creating an anemic condition.

To the best of applicant's knowledge, to date there has been no known effective means of arresting sickle cell anemia so as to prevent an individual who has this malady from experiencing one of the above-described problems. One known laboratory test employed in diagnosing sickle cell anemia is the performance of a hemoglobin electrophoresis test which is used to determine whether an individual has sickle cell anemia (homozygous) or merely the sickle cell trait (heterozygous), with the latter referring to an individual not having the disease but having the capability of transmitting the disease to offspring if mated to another heterozygote. Treatment for the various complications which have resulted from sickle cell anemia are known, and may be distinguished from prophylactic activity (unknown) which would resist the occurrence of the complications. Currently, only symptomatic treatment is readily available for those afflicted with this disease. For example, people can treat the symptoms by using analgesics for pain, and antibiotics for infection, but these approaches do not arrest the sickling phenomena.

Thus, there remains a very real and substantial need for a method of minimizing the adverse consequences of sickle cell anemia in subjects through resisting sickle cell crises in an individual who has this abnormality using compositions comprising sulfated polysaccharides, particularly non-animal derived sulfated polysaccharides, as described in further detail herein.

B. SPs as Therapeutic Agents.

Sulfated polysaccharides (SPS) typically have Heparin-like structures and can be absorbed directly to the endothelium of a subject via multiple routes of administration. SPS incorporate into the endothelial glycocalyx and diffuse to other cell glycocalyses for therapeutic effects. As a consequence of their polymeric structure, and particularly the sulfate and other charged groups decorating them, they interact with charged groups of various peptides and other molecules in manners analogous to those of endogenous GAG domains thereby increasing the regulatory and homeostatic properties of the glycocalyx. Upon incorporation into the glycocalyx SPS decrease the interaction of glycocalyx components with heparanases, proteases, and other GC degrading factors thereby preserving the integrity and function of the endogenous glycocalyx of endothelial and other cell types. Additionally, SPS possess anti-oxidant and other reactive metabolite activities that increases bioactivity and bioavailability of glycocalyx-associated Nitric Oxide and reduce cellular injury in various cells and tissues.

By these and other effects, it is thought that SPS, particularly those compositions described herein containing sulfated polysaccharides, have therapeutic effects in repairing and maintaining the glycocalyx of a subject, affecting the macromolecular permeability of LDL's, such as important in atherosclerotic diseases, metabolic diseases and disorders, diabetes, Sickle Cell Disease and other vasculopathies associated with glycocalyx destruction in endothelial and other cells and tissues. As a result of incorporation into and effects upon the endogenous glycocalyx of various cells, numerous beneficial effects to patients treated with non-animal SPs compositions, including but not limited to: increased anti-thrombotic and fibrinolytic effects; lower glycocalyx and cellular permeability for macromolecules of various size and structure; decreased inflammation and leucocyte, platelet, and monocytic attachment and adhesion due to selectin exposure and other adhesive processes; increased endogenous GC synthesis of functional polysaccharides; increased Nitric Oxide bioavailability and homeostasis effects; increased resistance to effects of reactive oxygen, nitrogen, and other reactive metabolites; decreased vaso-occlusive and ischemia/reperfusion effects on cells and tissues; and decreased endothelial cell senescence and increased endothelial repair.

Further, and while not wishing to be confined to theory, there is a chemical similarity between the sulfated polysaccharides heparin and rhamnan sulfate, suggesting evidence of analogous biological activity. Significantly, rhamnan sulfate (unlike heparin) has oral bioactivity. Several facts thus make rhamnan sulfate, and similar sulfated polysaccharides, viable drug candidates with potential clinical efficacy both as a surface-based, anti-thrombic agent, and as an LDL cholesterol permeability inhibitor. These effects, based on the experimental evidence presented herein, appear related to the bioactivity of rhamnan sulfate inducing the increased production of endogenous heparan sulfate and the incorporation into the endothelial glycocalyx. Additionally, following oral administration, rhamnan sulfate appears to localize to both arterial and venous endothelium in a dose-related manner.

Still further therapeutic targets for the use of the SPs detailed herein, and compositions containing such compounds, include the treatment of small vessel diseases such as retinopathy and nephropathy, including diabetic retinopathy; arteriosclerosis; immune disorders; treatment of vascular events (coronary heart disease (CHD), stroke, and heart disease); neurodegenerative disorders, including autism; and, cancer therapeutic applications, for the treatment of cancer in subjects.

Sulfated Polysaccharides.

Compositions according to the present invention include one or more naturally-occurring sulfated polysaccharide. Preferably, such sulfated polysaccharides are non-animal derived, meaning that the sulfated polysaccharides are derived from non-mammalian sources, such as plants, yeasts, fungi, algae, and microorganisms. Sulfated polysaccharides (SPs) for use in the methods of the present invention are sulfated polysaccharides that exhibit therapeutic activity in patients to which they are administered, and for which treatment of an appropriate disorder is needed. The non-coagulant properties of potential SPs are determined using a variety of assays, as set out herein above.

Exemplary sulfated polysaccharides suitable for use herein include those sulfated polysaccharides having the general structural formula (I),

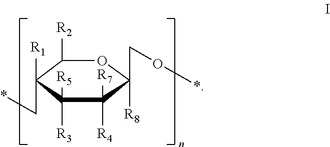

or a solvate, salt, prodrug, hydrate, derivative, or analog thereof,
wherein
R₁, R₂, R₃, R₄, R₅, R₆, R₇ and R₈ are either the same or different, and are independently selected from the following groups consisting of hydroxyl, thiol, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyoxyalkylene, polyol alkyl, alkylcarbonylalkyl, lower alkyl S(O)-lower alkyl, lower alkyl-S(O)₂-lower alkyl, aralkyl lower thioalkyl, heteroaralkyl lower thioalkyl, heterocyclealkyl lower thioalkyl, heteroaryl lower alkyl, heterocycle lower alkyl, heteroarylthio lower alkyl, arylthio lower alkyl, heterocyclethio lower alkyl, heteroarylamino lower alkyl, heterocycleamino lower alkyl, arylsulfinyl lower alkyl, and arylsulfonyl lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, alkylthio, haloalkylthio, thioether, amino, alkylamino, dialkylamino, alkylsulfonylamino, acylamino, arylamino, heteroarylamino, heterocycleamino, oxyalkylamino, amido, imide, sulfonylimide, carboxamido, sulfonamido, amino acid, amino acid esters, amino acid amides, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, hydroxamic acid, tetrazolyl, carbohydrate, or alditol; and
n is an integer selected from 1 to 20,000, inclusive, and
wherein the points of chirality (*) within each residue may be alpha (α), beta (β), or alternating alpha and beta between residues.

In accordance with further aspects of the present disclosure, sulfated polysaccharides suitable for use herein include those sulfated polysaccharides having the general structural formula (II),

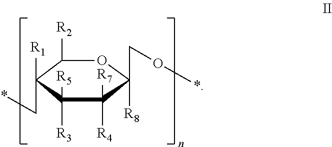

or a solvate, prodrug, hydrate, derivative, salt, or analog thereof,
wherein
R₁, R₂, R₃, R₄, R₅, R₆, R₇ and R₈ are either the same or different, and are independently selected from the following groups consisting of hydroxyl, thiol, halo, nitro, cyano, alkyl, alkenyl, alkynyl, aryl, carbocycle, haloalkyl, hydroxyalkyl, aminoalkyl, cycloalkyl, alkylcarbonylalkyl, lower alkyl S(O)-lower alkyl, lower alkyl-S(O)₂-lower alkyl, aralkyl lower thioalkyl, heteroaralkyl lower thioalkyl, heterocyclealkyl lower thioalkyl, heteroarylthio lower alkyl, arylthio lower alkyl, heterocyclethio lower alkyl, heteroarylamino lower alkyl, heterocycleamino lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, alkylthio, haloalkylthio, thioether, amino, alkylamino, dialkylamino, alkylsulfonylamino, acylamino, arylamino, heteroarylamino, heterocycleamino, oxyalkylamino, amido, imide, sulfonylimide, carboxamido, sulfonamido, amino acid, amino acid esters, amino acid amides, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, hydroxamic acid, tetrazolyl, carbohydrate, fucan, sorbitan, sugar, or alditol; and
n is an integer selected from 1 to 20,000, inclusive, and
wherein the points of chirality (*) within each residue may be alpha (α), beta (β), or alternating alpha and beta between residues.

In accordance with further aspects of the present disclosure, sulfated polysaccharides suitable for use herein include those sulfated polysaccharides having the general structural formula (III),

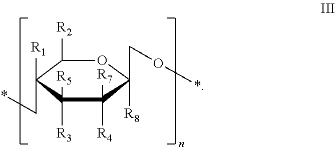

or a solvate, salt, prodrug, hydrate, derivative, or analog thereof,
wherein
is R₁, R₂, R₃, R₄, R₅, R₆, R₇ and R₈ are either the same or different,
at least one of R₁-R₈ is hydroxyl,
at least one of R₁-R₈ is OSO₃⁻M⁺, with M being selected from the group consisting of Na, K, Li, or H,
and the remaining residues are independently selected from the following groups consisting of hydroxyl, thiol, halo, nitro, cyano, alkyl, alkenyl, alkynyl, sulfonamido, amino acid, amino acid esters, amino acid amides, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, hydroxamic acid, tetrazolyl, carbohydrate, fucan, sorbitan, sugar, or alditol; and
n is an integer selected from 1 to 20,000, inclusive, and
wherein the points of chirality (*) within each residue may be alpha (α), beta (β), or alternating alpha and beta between residues.

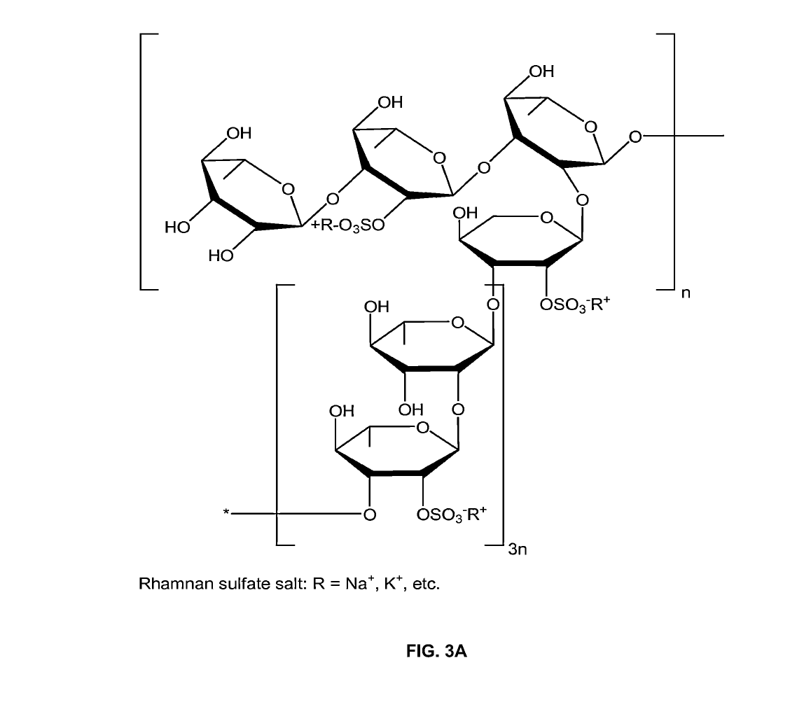

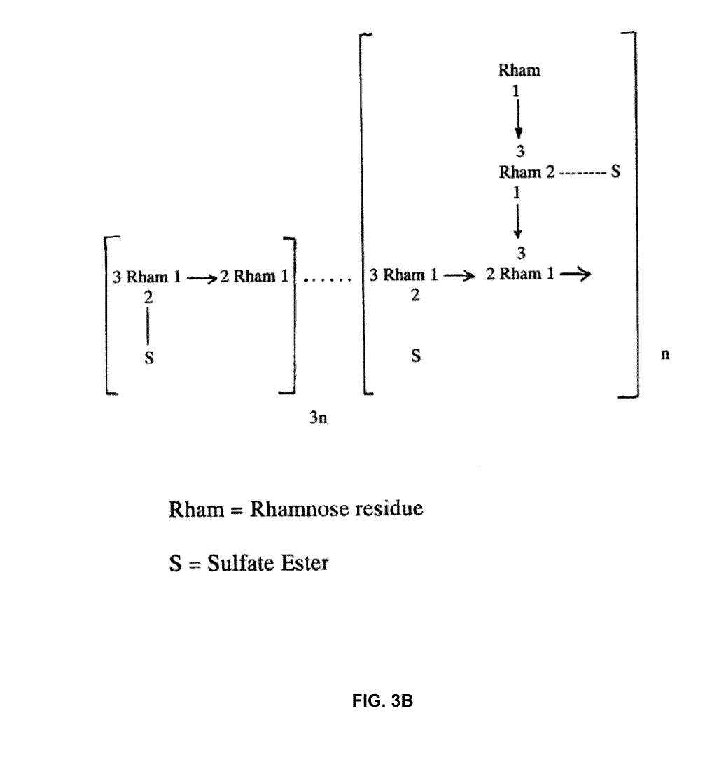

In accordance with further aspects of the present disclosure, the composition may comprise one or more sulfated polysaccharides having the general structure of rhamnan sulfate illustrated in FIG. 3A and FIG. 3B, as well as a solvate, salt, prodrug, hydrate, derivative, or analog thereof.

Sulfated polysaccharides with potential therapeutic activity in accordance with the present disclosure include, but are not limited to, glycosaminoglycans (GAGs), heparin-like molecules including N-acetyl heparin (Sigma-Aldrich, St. Louis, Mo.) and N-desulfated heparin (Sigma-Aldrich), sulfatoids, polysulfated oligosaccharides (Karst et al. (2003) Curr. Med. Chem. 10:1993-2031; Kuszmann et al. (2004) Pharmazie. 59:344-348), chondroitin sulfates (Sigma-Aldrich), dermatan sulfate (Celsus Laboratories Cincinnati, Ohio), fucoidan (Sigma-Aldrich), pentosan polysulfate (PPS) (Ortho-McNeil Pharmaceuticals, Raritan, N.J.), fucopyranon sulfates (Katzman et al. (1973) J. Biol. Chem. 248:50-55), and novel sulfatoids such as GM1474 (Williams et al. (1998) General Pharmacology 30:337) and SR 80258A (Burg et al. (1997) Laboratory Investigation 76:505), and novel heparinoids, and their analogs. NASPs may be purified and/or modified from natural sources (e.g. brown algae, tree bark, animal tissue) or may be synthesized de novo and may range in molecular weight from 100 daltons to 1,000,000 daltons. Additional compounds with potential NASP activity include periodate-oxidized heparin (POH) (Neoparin, Inc., San Leandro, Calif.), chemically sulfated laminarin (CSL) (Sigma-Aldrich), chemically sulfated alginic acid (CSAA) (Sigma-Aldrich), chemically sulfated pectin (CSP) (Sigma-Aldrich), dextran sulfate (DXS) (Sigma-Aldrich), heparin-derived oligosaccharides (HDO) (Neoparin, Inc., San Leandro, Calif.).

In principle, any free hydroxyl group on a monosaccharide component of a glycoconjugate can be modified by sulfation to produce a sulfated glycoconjugate for potential use as a SP in the practice of the invention. For example, such sulfated glycoconjugates may include without limitation sulfated mucopolysaccharides (D-glucosamine and D-glucoronic acid residues), curdlan (carboxymethyl ether, hydrogen sulfate, carboxymethylated curdlan) (Sigma-Aldrich), sulfated schizophyllan (Itoh et al. (1990) Int. J. Immunopharmacol. 12:225-223; Hirata et al. (1994) Pharm. Bull. 17:739-741), sulfated glycosaminoglycans, sulfated polysaccharide-peptidoglycan complex, sulfated alkyl malto-oligosaccharide (Katsuraya et al. (1994) Carbohydr Res. 260:51-61), amylopectin sulfate, N-acetyl-heparin (NAH) (Sigma-Aldrich), N-acetyl-de-O-sulfated-heparin (NA-de-b-SH) (Sigma-Aldrich), de-N-sulfated-heparin (De-NSH) (Sigma-Aldrich), and De-N-sulfated-acetylated-heparin (De-NSAH) (Sigma-Aldrich).

Other sulfated polysaccharides suitable for use herein include seaweed extracts, for example from brown and/or green algae and/or red algae, using any number of appropriate extraction methods, the only proviso associated with the method being that the polysaccharide, particularly the sulfated polysaccharide (e.g., Rhamnan sulfate, or the like) remain in the extract. Suitable brown algae that may be the source of the polysaccharide or sulfated polysaccharide may be selected from the group consisting of, but not limited to, Fucus vesiculosus, Laminaria brasiliensis, Laminaria saccharina, L. digitata, Analipus japonicas, Cladosiphon okamuranus, Chorda filum, Saccharina latissima, Turbinaria conoides, or Ascophylum nodosum. Exemplary suitable sulfated polysaccharides obtainable from brown algae for use in the compositions of the present disclosure include fucans—sulfated polysaccharides having a fucose (-Fuc) backbone, and which may optional also contain small amount of other monosaccharides, including but not limited to glucose, galactose, mannose, xylose, uronic acids, and/or acetyl groups. Green algae suitable as a source of polysaccharides or sulfated polysaccharides for use with the present disclosure may be selected from the group consisting of, but not limited to, green seaweed of the order Ulvales (Ulva and Enteromorpha sp., such as Ulva pertusa and U. lactuca) that have sulfate, rhamnose (Rha), xylose, iduronic (Ido) and glucuronic (Glc) acids as main constituents (e.g., ulvan, a water-soluble polysaccharide having as the main repeating disaccharide units ulvanobiouronic acid 3-sulfate types containing either glucuronic or iduronic acid), Monostroma nitidium, Monostroma zosteticola, Monostroma angicava, Monostroma lattissimum, Monostroma pulchrum, Monostroma fusem, Monostroma grevillei, Entoromorpha cornpressa, Ulva arasakii, Cladophora denna, Cladophora rugulosa, Chaecomorpha spiralis, Chaecomorpha crassa, Spongomorpha duriuscula, Codium fragile, Codium divaricaium Codium latum, or Caulerpa okamarai.

Exemplary sulfated polysacchardies may consist of 1,3-, 1,4, and 1,2-linked rhamnose residues in various ratios, including a ratio of 1:2, 3:1, and 3:2. In one aspect of the invention, the at least one naturally-occurring, sulfated polysaccharide is rhamnan sulfate (pure, as a salt, prodrug, or otherwise), particularly rhamnan sulfate obtained from M. nitidium or U. pertusa, or a mixture (e.g., 10 wt. %/90 wt. %-50 wt. %/50 wt. %) of rhamnan sulfates obtained or isolated from two different natural sources, e.g., two different algae or seaweed species. In some instances, sulfate may be substituted at C-3 or C-4 in the 1,2-linked rhamnose residue. In accordance with one aspect of the present disclosure, an exemplary rhamnan sulfate (RS)SPS consists of sugar linkage units as follows (1→3)-α-I-Rha-(1→3)-α-I-Rha-(1→2:→3)-α-I-Rha-(1→2)-α-I-Rha-(1→3:→3)-α-I-Rha-(1→3)-α-I-Rha-(1→3). In accordance with other aspects of the invention, sulfated polysaccharides containing primarily (greater than 60%) (1→2) linked 4-O-sulfated fucopyranose residues which may be branched or unbranched are suitable for use as sulfated polysaccharides.

Applications.

In one aspect, SPs may be used in the methods of the invention for treating disorders associated with the glycocalyx of a patient or subject, particularly those associated with disorders that can be linked to the glycocalyx of the subject, or for reversing the effects of glycocalyx dysfunction or destruction in a subject. For example, and without limitation, SPs may be administered to a subject to treat bleeding disorders, including congenital coagulation disorders, acquired coagulation disorders, and hemorrhagic conditions induced by trauma. Examples of other disorders that may be treated with SPs in accordance with the present disclosure include, but are not limited to, the in vivo effect on endothelial glycocalyx composition and function, reduction of vaso-occlusive crises, increased anti-thrombotic/fibrinolytic potential, and cellular anti-oxidant effects in sickle cell diseases; the reduction of glycocalyx permeability, composition, and function in vivo in familial dyslipidemia and diabetes patients; metabolic syndromes as described herein; and, the inhibition of glycocalyx degradation in atherothrombosis in a subject suffering from atherothrombosis.

Pharmaceutical Compositions.

The SP compositions of the present disclosure may be formulated to be substantially all active ingredient, with the remainder of the composition being inactive ingredients. Optionally, the SP compositions of the present invention may further comprise one or more pharmaceutically acceptable excipients to provide a pharmaceutical composition. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerine, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

A composition of the present invention can also include an antimicrobial agent for preventing or deterring microbial growth. Nonlimiting examples of antimicrobial agents suitable for the present invention include but are not limited to benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the composition as well. Antioxidants are used to prevent oxidation, thereby preventing the deterioration of the NASP or other components of the preparation. Suitable antioxidants for use in the present invention include, for example and without limitation, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant can be present as an excipient. Exemplary surfactants include: polysorbates, such as “Tween 20” and “Tween 80,” and pluronics such as F68 and F88 (BASF, Mount Olive, N.J.); sorbitan esters; lipids, such as phospholipids such as lecithin and other phosphatidylcholines, phosphatidylethanolamines (although preferably not in liposomal form), fatty acids and fatty esters; steroids, such as cholesterol; chelating agents, such as EDTA; and zinc and other such suitable cations.

Acids or bases can be present as an excipient in the composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, for example and without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the SP (e.g., when contained in a drug delivery system) in the composition will vary depending on a number of factors, but will optimally be a therapeutically effective dose when the composition is in a unit dosage form or container (e.g., a vial). A therapeutically effective dose can be determined experimentally by repeated administration of increasing amounts of the composition in order to determine which amount produces a clinically desired endpoint.

The amount of any individual excipient in the composition will vary depending on the nature and function of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight most preferred. These foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, N.J. (1998), and Kibbe, A. H., Handbook of Pharmaceutical Excipients, 3rd Edition, American Pharmaceutical Association, Washington, D.C., 2000.

The compositions encompass all types of formulations and in particular those that are suited for injection, e.g., powders or lyophilates that can be reconstituted with a solvent prior to use, as well as ready for injection solutions or suspensions, dry insoluble compositions for combination with a vehicle prior to use, and emulsions and liquid concentrates for dilution prior to administration. Examples of suitable diluents for reconstituting solid compositions prior to injection include bacteriostatic water for injection, dextrose 5% in water, phosphate buffered saline, Ringer's solution, saline, sterile water, deionized water, and combinations thereof. With respect to liquid pharmaceutical compositions, solutions and suspensions are envisioned. Additional preferred compositions include those for oral, ocular, or localized delivery.

Vitamin B₃ Compounds

The compositions of the present invention may contain a safe and effective amount of one or more vitamin B₃ compounds. When vitamin B₃ compounds are present in the compositions of the instant invention, the compositions can contain from about 0.01% to about 50%, or from about 0.1% to about 10%, or from about 0.5% to about 10%, or from about 1% to about 5%, or from about 2% to about 5%, by weight of the composition, of the vitamin B₃ compound.

As used herein, “vitamin B₃ compound” means a compound having the general formula IV, below:

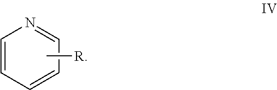

wherein R is —CONH₂ (e.g., niacinamide), —COOH (e.g., nicotinic acid) or —CH₂OH (e.g., nicotinyl alcohol), derivatives thereof, and salts of any of the foregoing. Exemplary derivatives of the foregoing vitamin B₃ compounds include nicotinic acid esters, including non-vasodilating esters of nicotinic acid (e.g., tocopheryl nicotinate), nicotinyl amino acids, nicotinyl alcohol esters of carboxylic acids, nicotinic acid N-oxide and niacinamide N-oxide.

The vitamin compounds may be included as the substantially pure material, or as an extract obtained by suitable physical and/or chemical isolation from natural (e.g., plant) sources.

Examples of suitable vitamin B₃ compounds for use with the compositions and formulations of the present disclosure are well known in the art and are commercially available from a number of commercial sources, including but not limited to, the Sigma Chemical Company (St. Louis, Mo.); ICN Biomedicals, Inc. (Irvin, Calif.) and Aldrich Chemical Company (Milwaukee, Wis.).

Vitamin B₅ Compounds

The compositions of the present invention may contain a safe and effective amount of a vitamin B₅ compound. When vitamin B₅ compounds are present in the compositions of the instant invention, the compositions can contain from about 0.01% to about 50%, or from about 0.1% to about 10%, or from about 0.5% to about 10%, or from about 1% to about 5%, or from about 2% to about 5%, by weight of the composition, of the vitamin B₅ compound.

As used herein, “vitamin B₅ compound” means a compound having the general formula V, below:

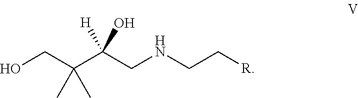

wherein R is OH, CO₂H, or S—S—(CH₂)₂NHC(O)CH₂(₂)—NHC(O)C(OH)CH(CH₃)₂CH₂OH (pantethine).

Vitamin C Compounds

The compositions of the present invention may contain a safe and effective amount of a vitamin C compound, or analog or derivative thereof, including vitamin C a vitamin C complex, or a mineral ascorbate. When a vitamin C compound is present in the composition of the present disclosure, it is preferred that it is present is an amount ranging from about 10 mg to about 500 mg per 55 kg of body weight of the animal, more preferably in an amount ranging from about 25 mg to about 400 mg per 55 kg of body weight of the animal, and more preferably in an amount ranging from about 25 mg to about 250 mg per 55 kg of body weight of the animal.

Vitamin E Compounds

The compositions of the present invention may also contain vitamin E (tocopherol) and/or one or more Vitamin E compounds or derivatives of tocopherol. The tocopherols employed in the present invention include α-tocopheraol, β-tocopherol, γ-tocopherol, and δ-tocopherol, as well as any of the known tocotrienols and combinations thereof, as well as derivatives of tocopherol of the structure VI, below:

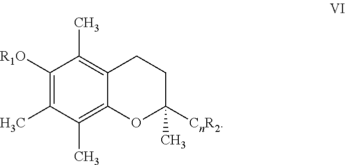

wherein n is an integer from 6 to 13, including 7, 8, 9, 10, 11, and 12; R₁ is hydrogen, alkyl, alkenyl, an ether, a silyl ether, or acetate; and R₂ is an optionally substituted nitrogen-containing heterocycle or a polycyclic nitrogen-containing heterocycle and pharmaceutically acceptable salts thereof. Exemplary tocopherol derivatives suitable for use in the compositions described herein include, but are not limited to, selected from the group consisting of (R)-2-(9-(1H-imidazol-1-yl)nonyl)-2,5,7,8-tetramethylchroman-6-ol; (R)-1-(9-(6-(tert-butyldimethylsilyloxy)-2,5,7,8 tetramethylchroman-2-yl)nonyl)-1H-1,2,3-triazole; (R)-1-(9-(6-(tert-butyldimethylsilyloxy)-2,5,7,8-tetramethylchroman-2-yl)-nonyl)-1H-1,2,4-triazole; (R)-2-(9-(1H-1,2,4-triazol-1-yl)nonyl)-2,5,7,8-tetramethyl-chroman-6-ol; (R)-2-(9-(1H-1,2,3-triazol-1-yl)nonyl)-2,5,7,8-tetramethylchroman-6-ol; (R)-2-(9-(6-(tert-butyldimethylsilyloxy)-2,5,7,8-tetramethylchroman-2-yl)-nonyl)-2H-1,2,3-triazole; (R)-2-(9-(2H-1,2,3-triazol-2-yl)nonyl)-2,5,7,8-tetramethylchroman-6-ol; (R)-1-(9-(6-(tert-butyldimethylsilyloxy)-2,5,7,8-tetramethylchroman-2-yl)-nonyl)-1H-benzo[d]imidazole; (R)-2-(9-(1H-benzo[d]imidazol-1-yl)nonyl)-2,5,7,8-tetramethyl-chroman-6-ol-; (R)-2-(9-(6-(tert-butyldimethylsilyloxy)-2,5,7,8-tetramethylchroman-2-yl-)nonyl)-2H-benzo[d][1,2,3]triazole; (R)-2-(9-(2H-benzo[d][1,2,3]triazol-2-yl)nonyl)-2,5,7,8-tetramethylchroma-n-6-ol; (R)-1-(9-(6-(tert-butyldimethylsilyloxy)-2,5,7,8-tetramethylchroma-n-2-yl)nonyl)-1H-benzo[d][1,2,3]triazole; (R)-2-(9-(1H-benzo[d][1,2,3]triazol-1-yl)nonyl)-2,5,7,8-tetramethylchroma-n-6-ol; 1-{9-[(R)-6-hydroxy-2,5,7,8-tetramethyl-chroman-2-yl]-nonyl}-5H-pyrimidine; and 1-{9-[(R)-6-hydroxy-2,5,7,8-tetramethyl-chroman-2-yl]-nonyl}-2H-pyrazine.

The vitamin E compounds present in the compositions of the instant invention are in an amount ranging from about 0.01% to about 20%, or from about 0.1% to about 10%, or from about 0.5% to about 10%, or from about 1% to about 5%, or from about 2% to about 5%, by weight of the composition, of the tocopherol, vitamin E compound.

Amino Acids

Amino acids, including but not limited to alpha-amino acids, beta-amino acids, and derivatives and analogs thereof, in both the L- and D-form (as appropriate) may be included in the compositions of the present invention in safe and effective amounts. As used herein, the term “amino acid” refers to both naturally-occurring and synthetic amino acids, including both alpha- and beta-amino acids. Exemplary amino acids suitable for use in the compositions described herein include but are not limited to L-arginine, L-aspartamine, aspartic acid, L-proline, L-serine, L-tyrosine, L-tryptophan, L-lysine, L-glycine, L-leucine, L-alanine, L-phenylalanine, L-valine, L-cysteine, L-methionine, and L-glutamine. Most preferably, the compositions of the present invention include at least the amino acid L-arginine or a similar sulfur-containing amino acid such as an oligomer of L-arginine (e.g., from 2 to 20 residues of L-arginine or an L-arginine analog, or from 2 to 20 residues of L-arginine or an L-arginine analog, or from 2 to 10 residues of L-arginine or an L-arginine analog), an L-arginine analog, or an L-arginine analog oligomer linked through a labile bond to a polymeric matrix, in a therapeutically effective amount ranging from about 0.1 to about 15.0 wt. %, by weight of the total composition.

Peptides

Peptides, including but not limited to, di-, tri-, tetra-, and pentapeptides and derivatives thereof, may be included in the compositions and formulations of the present invention in amounts that are safe and effective. As used herein, “peptides” refers to both the naturally occurring peptides and synthesized peptides, as well as peptidomimetics. Also useful herein are naturally occurring and commercially available compositions that contain peptides.

Suitable dipeptides for use herein include but are not limited to Carnosine (beta-ala-his). Suitable tripeptides for use herein include, gly-his-lys, arg-lys-arg, and his-gly-gly. Preferred tripeptides and derivatives thereof include palmitoyl-gly-his-lys, which may be purchased as Biopeptide CL™ (100 ppm of palmitoyl-gly-his-lys commercially available from Sedenna, France); Peptide CK (arg-lys-arg); Peptide CK+ (ac-arg-lys-arg-NH₂); and a copper derivative of his-gly-gly sold commercially as lamin, from Sigma (St. Louis, Mo.).

When included in the present compositions, peptides can be present in amounts ranging from about 1×10⁻⁶% to about 10% by total weight of the composition, or from about 1×10⁻⁶% to about 0.1% by total weight of the composition, or from about 1×10⁻⁵% to about 0.01% by total weight of the composition, by weight of the composition. In certain compositions, where the peptide is a specifically desired peptide (such as the Arg-Gly-Asp tripeptide), the compositions can contain from about 0.1% to about 5%, by weight of the composition, of such peptides. In other embodiments wherein the peptide-containing compositions, MATRIXYL™, and/or Biopeptide CL™ are included, the compositions can contain from about 0.1% to about 10%, by weight compositions, of MATRIXYL™ and/or Biopeptide CL™ peptide-containing compositions.

According to the present invention, the therapeutic compositions described herein are provided in a composition, e.g., pharmaceutical, medicinal or comestible composition, or alternatively in a formulation, e.g., a pharmaceutical or medicinal formulation or a food or beverage product or formulation.

Commonly used known or artificial sweeteners for use in such combinations of sweeteners and the therapeutic agent include but are not limited to the common saccharide sweeteners, e.g., sucrose, fructose, glucose, and sweetener compositions comprising those natural sugars, such as corn syrup or other syrups or sweetener concentrates derived from natural fruit and vegetable sources, or semi-synthetic “sugar alcohol” sweeteners such as erythritol, isomalt, lactitol, mannitol, sorbitol, xylitol, maltodextrin, and the like, or well-known artificial sweeteners such as aspartame, saccharin, acesulfame-K, cyclamate, sucralose, and alitame; or any mixture thereof.

The therapeutic compositions of the present invention can also be provided, individually or in combination, with any comestible composition known or later discovered. A variety of classes, subclasses and species of comestible compositions are known. Exemplary comestible compositions include one or more confectioneries, chocolate confectionery, tablets, countlines, bagged selflines/softlines, boxed assortments, standard boxed assortments, twist wrapped miniatures, seasonal chocolate, chocolate with toys, alfajores, other chocolate confectionery, mints, standard mints, power mints, boiled sweets, pastilles, gums, jellies and chews, toffees, caramels and nougat, medicated confectionery, lollipops, liquorice, other sugar confectionery, gum, chewing gum, sugarized gum, sugar-free gum, functional gum, bubble gum, bread, packaged/industrial bread, unpackaged/artisanal bread, pastries, cakes, packaged/industrial cakes, unpackaged/artisanal cakes, cookies, chocolate coated biscuits, sandwich biscuits, filled biscuits, savory biscuits and crackers, bread substitutes, breakfast cereals, rte cereals, family breakfast cereals, flakes, muesli, other cereals, children's breakfast cereals, hot cereals, ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yoghurt, artisanal ice cream, dairy products, milk, fresh/pasteurized milk, full fat fresh/pasteurized milk, semi skimmed fresh/pasteurized milk, long-life/uht milk, full fat long life/uht milk, semi skimmed long life/uht milk, fat-free long life/uht milk, goat milk, condensed/evaporated milk, plain condensed/evaporated milk, flavored, functional and other condensed milk, flavored milk drinks, dairy only flavored milk drinks, flavored milk drinks with fruit juice, soy milk, sour milk drinks, fermented dairy drinks, coffee whiteners, powder milk, flavored powder milk drinks, cream, cheese, processed cheese, spreadable processed cheese, unspreadable processed cheese, unprocessed cheese, spreadable unprocessed cheese, hard cheese, packaged hard cheese, unpackaged hard cheese, yoghurt, plain/natural yoghurt, flavored yoghurt, fruited yoghurt, probiotic yoghurt, drinking yoghurt, regular drinking yoghurt, probiotic drinking yoghurt, chilled and shelf-stable desserts, dairy-based desserts, soy-based desserts, chilled snacks, fromage frais and quark, plain fromage frais and quark, flavored fromage frais and quark, savory fromage frais and quark, sweet and savory snacks, fruit snacks, chips/crisps, extruded snacks, tortilla/corn chips, popcorn, pretzels, nuts, other sweet and savory snacks, snack bars, granola bars, breakfast bars, energy bars, fruit bars, other snack bars, meal replacement products, slimming products, convalescence drinks, ready meals, canned ready meals, frozen ready meals, dried ready meals, chilled ready meals, dinner mixes, frozen pizza, chilled pizza, soup, canned soup, dehydrated soup, instant soup, chilled soup, hot soup, frozen soup, pasta, canned pasta, dried pasta, chilled/fresh pasta, noodles, plain noodles, instant noodles, cups/bowl instant noodles, pouch instant noodles, chilled noodles, snack noodles, canned food, canned meat and meat products, canned fish/seafood, canned vegetables, canned tomatoes, canned beans, canned fruit, canned ready meals, canned soup, canned pasta, other canned foods, frozen food, frozen processed red meat, frozen processed poultry, frozen processed fish/seafood, frozen processed vegetables, frozen meat substitutes, frozen potatoes, oven baked potato chips, other oven baked potato products, non-oven frozen potatoes, frozen bakery products, frozen desserts, frozen ready meals, frozen pizza, frozen soup, frozen noodles, other frozen food, dried food, dessert mixes, dried ready meals, dehydrated soup, instant soup, dried pasta, plain noodles, instant noodles, cups/bowl instant noodles, pouch instant noodles, chilled food, chilled processed meats, chilled fish/seafood products, chilled processed fish, chilled coated fish, chilled smoked fish, chilled lunch kit, chilled ready meals, chilled pizza, chilled soup, chilled/fresh pasta, chilled noodles, oils and fats, olive oil, vegetable and seed oil, cooking fats, butter, margarine, spreadable oils and fats, functional spreadable oils and fats, sauces, dressings and condiments, tomato pastes and purees, bouillon/stock cubes, stock cubes, gravy granules, liquid stocks and fonds, herbs and spices, fermented sauces, soy based sauces, pasta sauces, wet sauces, dry sauces/powder mixes, ketchup, mayonnaise, regular mayonnaise, mustard, salad dressings, regular salad dressings, low fat salad dressings, vinaigrettes, dips, pickled products, other sauces, dressings and condiments, baby food, milk formula, standard milk formula, follow-on milk formula, toddler milk formula, hypoallergenic milk formula, prepared baby food, dried baby food, other baby food, spreads, jams and preserves, honey, chocolate spreads, nut-based spreads, and yeast-based spreads.

Exemplary comestible compositions also include confectioneries, bakery products, ice creams, dairy products, sweet and savory snacks, snack bars, meal replacement products, ready meals, soups, pastas, noodles, canned foods, frozen foods, dried foods, chilled foods, oils and fats, baby foods, or spreads or a mixture thereof.

Exemplary comestible compositions also include ice creams, breakfast cereals, sweet beverages or solid or liquid concentrate compositions for preparing beverages, ideally so as to enable the reduction in concentration of previously known saccharide sweeteners, or artificial sweeteners.

In another embodiment, the therapeutic compositions of the present invention may be added to food or beverage products or formulations. Examples of food and beverage products or formulations include any entity described in the Wet Soup Category, the Dehydrated and Culinary Food Category, the Beverage Category, the Frozen Food Category, the Snack Food Category, and seasonings or seasoning blends.

In general, “Wet Soup Category” usually means wet/liquid soups regardless of concentration or container, including frozen Soups. For the purpose of this definition soup(s) means a food prepared from meat, poultry, fish, vegetables, grains, fruit and other ingredients, cooked in a liquid which may include visible pieces of some or all of these ingredients. It may be clear (as a broth) or thick (as a chowder), smooth, pureed or chunky, ready-to-serve, semi-condensed or condensed and may be served hot or cold, as a first course or as the main course of a meal or as a between meal snack (sipped like a beverage). Soup may be used as an ingredient for preparing other meal components and may range from broths (consomme) to sauces (cream or cheese-based soups).

“Dehydrated and Culinary Food Category” usually means: (i) Cooking aid products such as: powders, granules, pastes, concentrated liquid products, including concentrated bouillon, bouillon and bouillon like products in pressed cubes, tablets or powder or granulated form, which are sold separately as a finished product or as an ingredient within a product, sauces and recipe mixes (regardless of technology); (ii) Meal solutions products such as: dehydrated and freeze dried soups, including dehydrated soup mixes, dehydrated instant soups, dehydrated ready-to-cook soups, dehydrated or ambient preparations of ready-made dishes, meals and single serve entrees including pasta, potato and rice dishes; and (iii) Meal embellishment products such as: condiments, marinades, salad dressings, salad toppings, dips, breading, batter mixes, shelf stable spreads, barbecue sauces, liquid recipe mixes, concentrates, sauces or sauce mixes, including recipe mixes for salad, sold as a finished product or as an ingredient within a product, whether dehydrated, liquid or frozen.

“Beverage Category” typically means beverages, beverage mixes and concentrates, including but not limited to, alcoholic and non-alcoholic, ready to drink beverages, liquid concentrate formulations for preparing beverages such as sodas, and dry powdered beverage precursor mixes.

Other examples of food and beverage products or formulations include carbonated and non-carbonated beverages, e.g., sodas, fruit or vegetable juices, alcoholic and non-alcoholic beverages, confectionery products, e.g., cakes, cookies, pies, candies, chewing gums, gelatins, ice creams, sorbets, puddings, jams, jellies, salad dressings, and other condiments, cereal, and other breakfast foods, canned fruits and fruit sauces and the like. Exemplary food and beverage products or formulations also include sweet coatings, frostings, or glazes for comestible products.

In yet another embodiment, the therapeutic compositions can be formulated, individually or in combination, in flavor preparations to be added to food and beverage formulations or products.

Typically at least therapeutically effective amount of a sweet flavor or a sweet flavor modulating amount, in combination with a therapeutically effective amount of the SP's of the present invention, will be added to the comestible or medicinal product, optionally in the presence of known sweeteners, e.g., so that the sweet flavor modified comestible or medicinal product has an increased sweet taste as compared to the comestible or medicinal product prepared without the modifiers of the present invention, as judged by human beings or animals in general, or in the case of formulations testing, as judged by a majority of a panel of at least eight human taste testers, via procedures commonly known in the field.

The concentration of sweet flavoring agent needed to modulate or improve the flavor of the comestible or medicinal product or composition will of course depend on many variables, including the specific type of comestible composition and its various other ingredients, especially the presence of other known sweet flavoring agents and the concentrations thereof, the natural genetic variability and individual preferences and health conditions of various human beings tasting the compositions, and the subjective effect of the particular compound on the taste of such chemosensory compounds.

In yet another embodiment, the therapeutic compositions of the present invention can be provided in pharmaceutical compositions containing a therapeutically effective amount of one or more compounds of the present invention, preferably in purified form, together with a suitable amount of a pharmaceutically acceptable vehicle, so as to provide the form for proper administration to a patient.

When administered to a patient, the compounds of the present invention and pharmaceutically acceptable vehicles are preferably sterile. Water is a preferred vehicle when a compound of the present invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present pharmaceutical compositions, if desired, can also contain minor amounts of wetting or emulsifying agents, or pH buffering agents. In addition, auxiliary, stabilizing, thickening, lubricating and coloring agents may be used.

Pharmaceutical compositions comprising a compound of the present invention may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries, which facilitate processing of compounds of the present invention into preparations which can be used pharmaceutically. Proper formulation is dependent upon the route of administration chosen.

The present pharmaceutical compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In some embodiments, the pharmaceutically acceptable vehicle is a capsule (see e.g., Grosswald et al., U.S. Pat. No. 5,698,155). Other examples of suitable pharmaceutical vehicles have been described in the art (see Remington: The Science and Practice of Pharmacy, Philadelphia College of Pharmacy and Science, 20^{th} Edition, 2000).

For topical administration a compound of the present invention may be formulated as solutions, gels, ointments, creams, suspensions, etc. as is well-known in the art.

Systemic formulations include those designed for administration by injection, e.g., subcutaneous, intravenous, intramuscular, intrathecal or intraperitoneal injection, as well as those designed for transdermal, transmucosal, oral or pulmonary administration. Systemic formulations may be made in combination with a further active agent that improves mucociliary clearance of airway mucus or reduces mucous viscosity. These active agents include, but are not limited to, sodium channel blockers, antibiotics, N-acetyl cysteine, homocysteine and phospholipids.

In some embodiments, the compounds of the present invention are formulated in accordance with routine procedures as a pharmaceutical composition adapted for intravenous administration to human beings. Typically, compounds of the present invention for intravenous administration are solutions in sterile isotonic aqueous buffer. For injection, a compound of the present invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents. When necessary, the pharmaceutical compositions may also include a solubilizing agent.

Administration.

At least one therapeutically effective cycle of treatment with an SP in accordance with the present disclosure will be administered to a subject. By “therapeutically effective cycle of treatment” is intended a cycle of treatment that when administered, brings about a positive therapeutic response with respect to treatment of an individual for a bleeding disorder. Of particular interest is a cycle of treatment with a sulfated polysaccharide that provide a positive therapeutic response in a patient suffering from a disorder as discussed herein, particularly an anemia, hemoglobin, or sickle cell disorder. By “positive therapeutic response” is intended that the individual undergoing treatment according to the invention exhibits an improvement in one or more symptoms of a bleeding disorder, including such improvements as shortened blood clotting times and reduced bleeding and/or reduced need for factor replacement therapy.

Exemplary manners of delivery of the therapeutic compositions described herein include, but are not limited to, oral and sublingual (such as a NTG-RS sublingual preparation), arenteral (IV or IM), aerosol administration (nasal or pulmonary), transdermal, or via devices, including but not limited to stents, implantable reservoirs, SPS synthesis chips or devices with assembly SPS enzymes embedded within them, synthesis devices, chips, or nano devices, and the like.

In certain embodiments, multiple therapeutically effective doses of compositions comprising one or more SPs and/or other therapeutic agents may be administered, over an appropriate period of time (e.g., hours, days, weeks, months, or years), as appropriate in order to obtain the desired therapeutic effect.

The following examples are included to demonstrate preferred embodiments of the inventions. It should be appreciated by those of skill in the art that the techniques disclosed in the examples which follow represent techniques discovered by the inventors to function well in the practice of the inventions, and thus can be considered to constitute preferred modes for its practice. However, those of skill in the art should, in light of the present disclosure, appreciate that many changes can be made in the specific embodiments which are disclosed and still obtain a like or similar result without departing from the scope of the inventions.

### EXAMPLES

General Materials and Methods.

In the experiments described herein, four sulfated polysaccharides were used, indicated as RS #1, RS #2, RS #3, and RS #4. RS #1 is the L-arginine (L-Arg) salt of rhamnan sulfate derived from Monostroma Nitidum. It was prepared by dialyzing out sodium ions and then re-binding the negative sulfate groups to the positively charged amino acid L-arginine, as detailed below. RS #2 is pure rhamnan sulfate from M. Nitidum, and physiologically may be considered to be a sodium salt as referenced above. RS #3 is rhamnan sulfate obtained from Ulva pertusa. RS #4 is a mixture (50/50 wt. %) of rhamnan sulfates from both Ulva pertusa and Ulva lactuca.

FTIR Analysis of Seaweed Samples.

Seaweed mixed with dry ice was ground to a fine powder using a mortar and pestle. The resulting material was dried exhaustively using a freeze drier. The resulting plant tissue was mixed with KBr and both were ground together in a mortar and pestle and then dried in a desiccator over P₂O₅. The dry salt-plant tissue mixture was pressed into a pellet and the FTIR spectrum was determined.

Water Extraction of Rhamnan Sulfate from Seaweed.

The dried seaweed was swollen in 20 volume (w/v) of water at room temp for 1 hr, then it was blended and refluxed in a boiling-water bath for 2 hr. The water extract was centrifuged at 4500 g for 30 min. The water-soluble rhamnan sulfate in the supernatant was precipitated in 65% (v/v) ethanol at 4° C. over night. The separated crude rhamnan sulfate in the precipitation was freeze dried

FITR Analysis of Rhamnan Sulfate.

Rhamnan sulfate was mixed with KBR and both were ground together in a mortar and pestle and then dried in a desiccator over P₂O₅. The dry salt-rhamnan sulfate mixture was pressed into a pellet and the FTIR spectrum was determined.

NMR Analysis of Rhamnan Sulfate.

NMR spectroscopy was performed on samples (˜5 mg) dissolved in D₂O (99.96 atom %), filtered through a 0.45 μm syringe filter, freeze-dried twice from D₂O to remove exchangeable protons and transferred to an NMR tube. One dimensional (1D) ¹H-NMR experiments were performed on a Bruker DRX-400 equipped with NMR Nuts (PC computer) processing and plotting software.

PAGE Analysis of Rhamnan Sulfate.

Gradient polyacrylamide gel electrophoresis (PAGE) was performed on a 32 cm vertical slab gel Bio-Rad unit equipped with model 1420B power source from Bio-Rad (Richmond, Calif.). Polyacrylamide linear gradient resolving gels (14×28 cm, 12% acrylamide) was prepared and run as described previously (Toida, et al., 1997). The molecular sizes were determined by comparing with a banding ladder of heparin oligosaccharide standards prepared from bovine lung heparin with a tetrasaccharide marker added to identify the bands. The gel was visualized by silver staining (Al-Hakim and Linhardt, 1991). After staining with alcian blue, the gel was soaked several hours in 50% (v/v) aqueous methanol followed with distilled water for 2 h. Silver staining solution was freshly prepared by adding 2 ml of 4 M silver nitrate, 2 ml of 7.6 M sodium hydroxide solution and 2.8 ml of ammonium hydroxide solution to 193.8 ml of degassed glass distilled water. The gel was stained with gentle shaking for 1 h followed by 3 washes with distilled water over 30 min. The gel was developed in freshly prepared developing buffer prepared by adding 1 ml of 2.5% (w/v) citric acid and 250 μl of 38% (v/v) formaldehyde solution to 500 ml of distilled water. Bands start to develop within 1 to 15 min. The reaction was stopped by placing the gel in 5% (v/v) aqueous acetic acid solution containing 20% (v/v) methanol. Molecular weight determination was performed by polyacrylamide gel electrophoresis (Edens, et al., 1992) using oligosaccharide standards prepared from bovine lung heparin. The gel was analyzed using UBN-Scan-IT gel, automated digitizing system, version 4.3 for Macintosh from Silk Scientific Corp., CA, USA.

Preparation of Rhamnose Sulfate-Arginine Complex.

Rhamnan sulfate Na-form was transformed to protonic form in a column (2.6×16 cm, 65 ml) packed with DOWEX 50wx8-100 H+-form. Rhamnan sulfate Na-form (mL at: 25 mg/ml) was loaded on the column and eluted with water. The maximum capacity of DOWEX 50wx8-100 was determined to be 0.62 g rhamnan sulfate/ml resin. The pooled (fractions with pH<3.5) Rhamnan sulfate protonic form was then neutralized with 0.5 M arginine to pH 5.5. The resulting rhamnan-arginine product was freeze dried. The NMR was obtained as previously described.

### Example 1

### Disaccharide Analysis

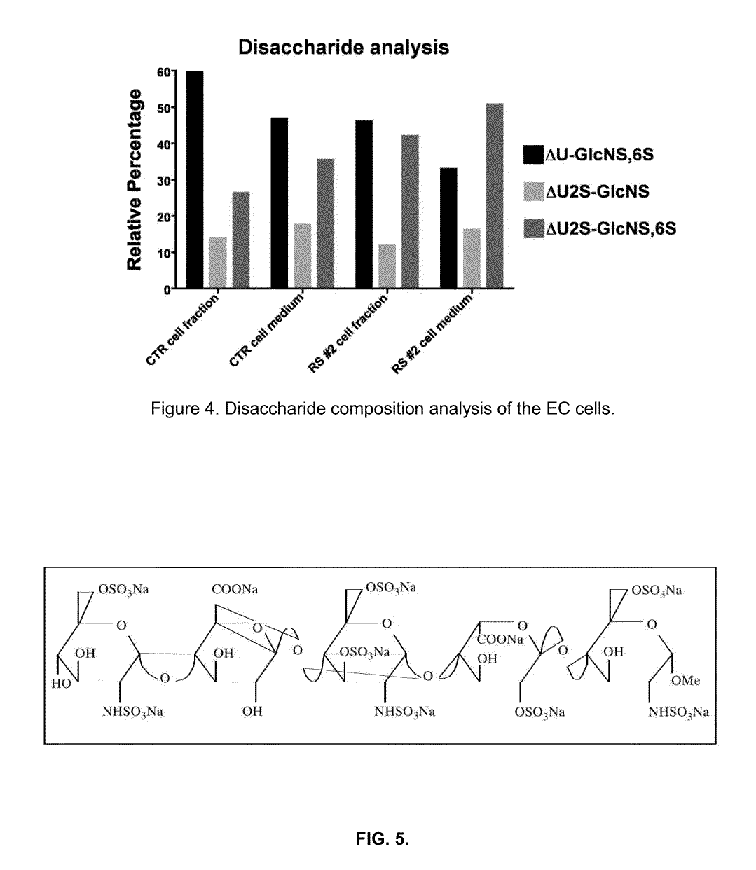

Purified heparin sulfate (HS) from the endothelial cells (EC) exposed or not exposed to RS #2 were incubated with a mixture of heparin lyases (heparinase I and II, 2.5 mlU each, available from Sigma) and the disaccharides produced by the enzymatic action were separated on a Phenosphere SAX column (Phenomenex, Torrance, Calif., USA) 150×4.6 mm using a NaCl gradient of 0-1 M in 30 min with a flux of 1 mL/min. Individual fractions (0.5 mL) were collected and counted using a micro-beta counter. FIG. 4 shows the relative percentage of the sulfated disaccharides, where CTR is the control cell fraction. As is apparent, the HS synthesized by the EC cells exposed to RS #2 show a significant increase in heparin sequences composed of the tri-sulfated disaccharide (ΔU2S-GlcNS,6S). The abbreviations used in the figure are: GlcNS(6S)=2-deoxy-6-O-sulfo-2-(sulfoamino)-α-D-glucopyranoside; GlcA=β-D-glucopyranuronosyl; GlcNS(3S,6S)=2-deoxy-3,6-di-O-sulfo-2-(sulfoamino)-α-D-glucopyranosyl; IdoA(2S)=2-O-sulfo-α-L-idopyranuronosyl; and, GlcNS(6S)OMe=methyl-O-2-deoxy-6-O-sulfo-2-(sulfoamino)-α-D-glucopyranoside.

The results of this figure also suggest that the RS increased the sulfation levels of the HS synthesized by the EC. As for the heparin stimulus, the HS produced by these cells have more of heparin sequences [-4) IdoA2S(1-4)GlcNS,6S] (heparin's basic structure). The results also show that the hepararin sulfate (HS) being induced is highly sulfated and has considerable structural analogy to low molecular weight (LMW) heparin and the drug Fondaparinux, shown generally in FIG. 5. Counting from the left, the RS-induced heparans are chains of disaccharide pairs like sugar groups number 4-5 hooked together. Additionally, the major fraction of stimulated heparan sulfate produced are tri-sulfated discaccharides (Iodo2S-GlcNS,6S), typical of the major sequence in heparin.

### Example 2

### LDL Permeability and Water Flux

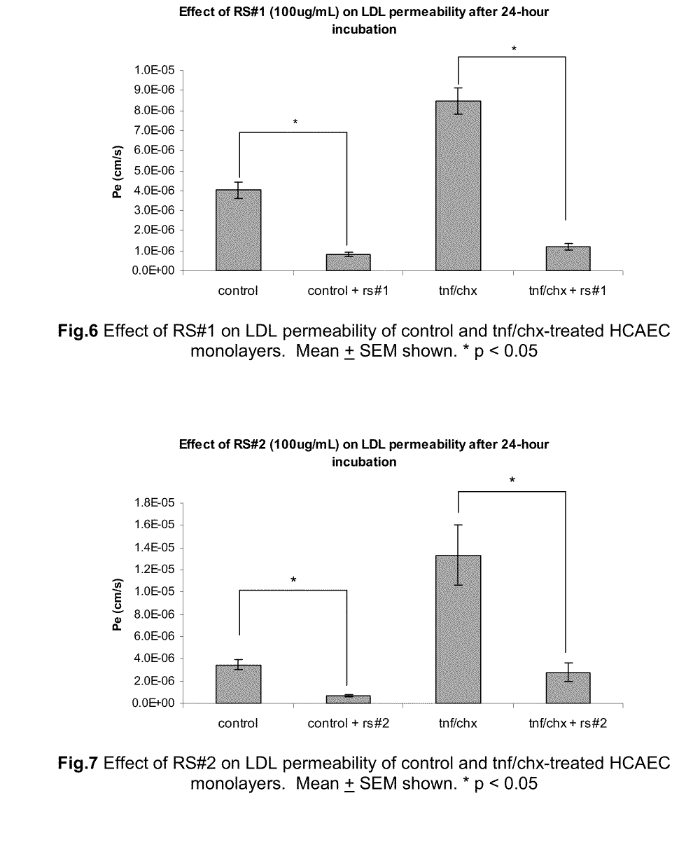

To test whether any of the RS isoforms could inhibit apoptosisn in human coronary artery endothelial cells (HCAEC), the HCAEC (human coronary artery endothelial cell) monolayers were incubated with each of two isoforms, RS#1 or RS#2, at concentrations ranging from 12.5 to 1200₄/mL for a period of time ranging from 24 hrs to 5 day, before the permeability of LDL (low density lipoprotein) was measured. In addition, some monolayers were induced to undergo apoptosis with TNF-α/CHX in the presence or absence of RS#1 or RS#2 and the permeability of LDL was measured. As shown in the figure, Dil means 11′-dioctadecyl-3,3-3′,3′-tetramethylindo-carbocyanide perchlorate. Both RS#1 and RS#2 significantly reduced the LDL permeability of control monolayers by 5-fold (FIGS. 6 and 7). When the monolayers were induced to undergo apoptosis the permeability increased by ˜2-3-fold. Incubation with RS#1 completely abolished the TNF-α/CHX-induced increase in permeability, bringing the permeability well below control levels (FIG. 6). Similarly, incubation with RS#2 completely abolished the TNF-α/CHX-induced increase in permeability, bringing the permeability down to control levels.

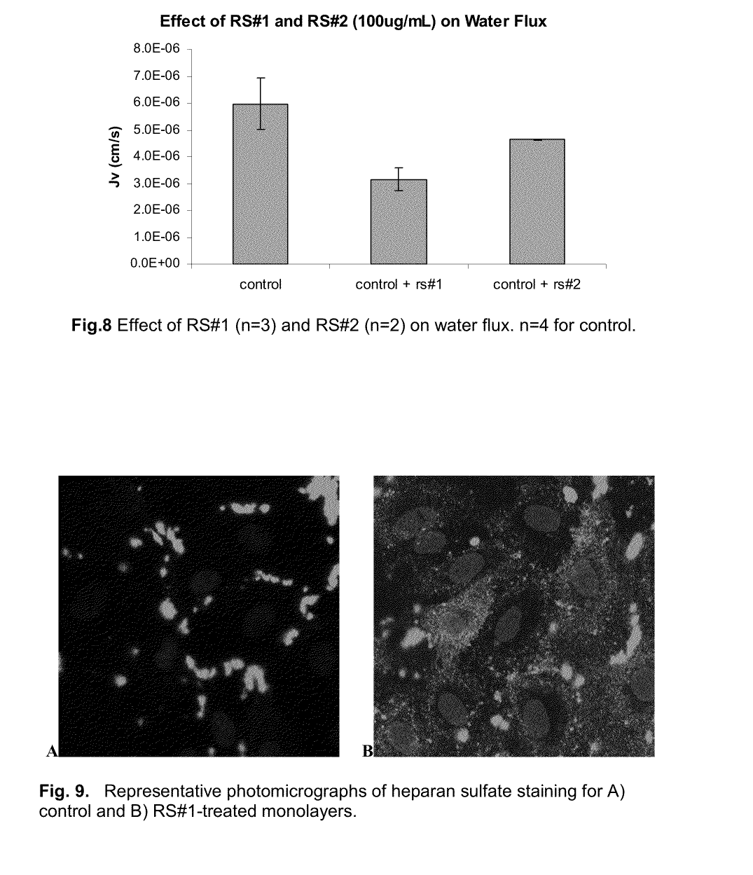

As shown in FIG. 8, the RS #1 isoform decreased the water flux through HCAEC monolayers by ˜50%, while the RS #2 isoform decreased the water flux by ˜20%. The increase in endothelial glycocalyx heaparan sulfate resulting from exposure to RS is associated with an 80% decrease in LDL cholesterol permeability as well as a significant reduction in albumin and water transport.

### Example 3

### Heparan Sulfate Immunostaining

HCAEC monolayers were incubated with RS#1 at 1004/mL for 24 hrs and immunostaining for heparan sulfate was performed. The monolayers were then imaged using a laser scanning confocal microscope. One control and one RS#1-treated monolayers were stained and four representative fields from each case were imaged. In control monolayers, heparan sulfate immunostaining seemed to be concentrated on the cell-cell junction area (FIG. 9A). Incubation to RS#1 increased the coverage of heparan sulfate on HCAEC monolayers (FIG. 9B). These results indicated that RS dramatically decreases LDL permeability even in the face of the very potent permeability enhancer and toxin TNF (tumor necrosis factor). They also show that this effect is related to ECM heparan production and localization at the endothelial cell-cell junctions.

These results also show that RS from Monostroma nitidum decreases permeability of LDL across the endothelium by ninety-three percent, an entirely unprecedented result. Lowering plasma LDL with statins does not improve permeability for LDL. This opens the prospect of co-encapsulating a statin for lowering LDL with RS that prevents LDL from entering the artery. Further, the observed decrease in LDL permeability appears by confocal microscopy related to trapping of LDL within the heparan sulfate-enhanced endothelial glycocalyx and reduced LDL transport across the endothelial cell wall.

### Example 4

### Heparan Sulfate Stimulus

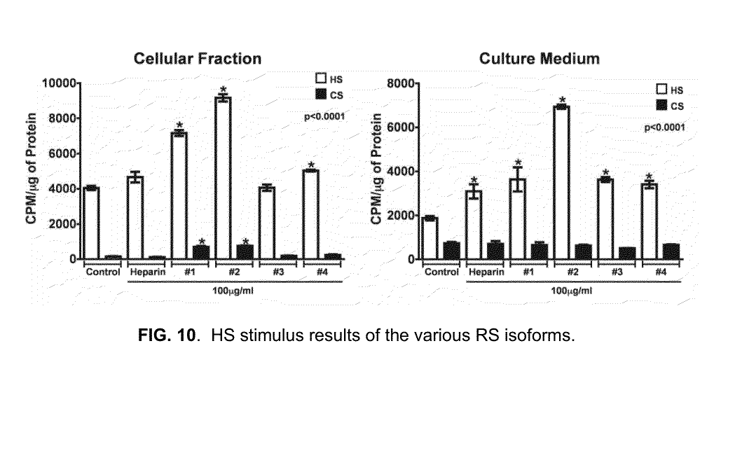

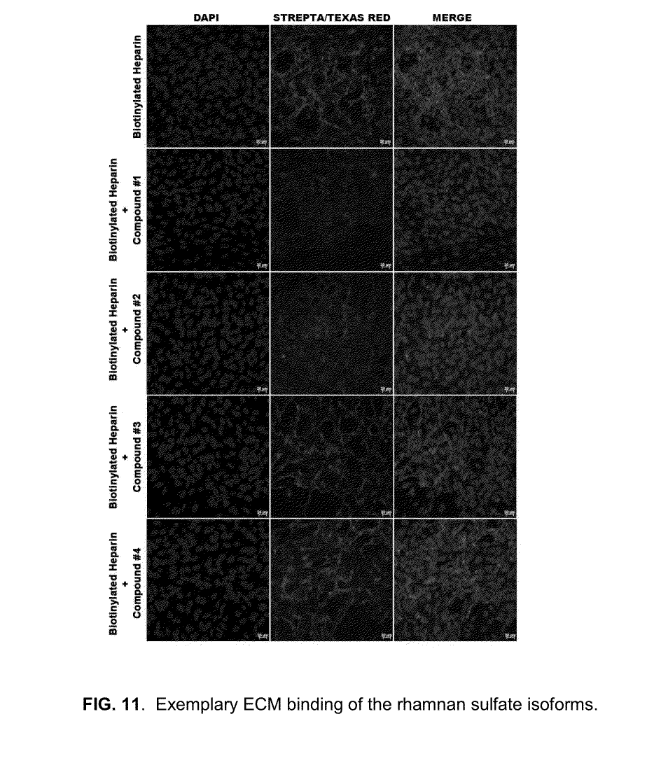

A heparin sulfate (HS) stimulus experiment was performed as known in the art, using the four RS isoforms described herein. As can be seen from the graphical results in FIG. 10, (the figure on the left showing cellular fraction of haparans in controls), it is notable that all four of the RS isoforms tested increased the release of heparans to cell medium as well as increased cellular HS content above control levels to differing levels. Number three shows that there is no change in cellular fraction of haparans resultant from the ulva pertusa RS isoform. Thus, ulva pertusa RS does not appear to increase the cellular content of haparans. These results also suggest that sulfated PS of a variety of origins can induce heparan and GAG (glycosamineoglycan) synthesis in endothelial cells and in other cells by processes of interstitial flow and diffusion. While the results shown here are limited to the RS isoforms studied, the type of polysaccharides to which this applies can be expanded to other classes of compounds that likely have similar effects, such as PUFAs, non-sulfated PS, other polymers, and the like. These RS (and other similar acting substances found in nature) can be used for in vivo HS and GAG production instead of administering HS or other GAGS for therapeutic effect.

Other findings based on the studies described herein suggest that rhamnan sulfate (RS) binds directly to the endothelial cell glycocalyx and interacts with heparan sulfate of the glycocalyx, and that RS significantly stimulates heparan sulfate production by endothelial cells.

Other and further embodiments utilizing one or more aspects of the inventions described above can be devised without departing from the spirit of Applicant's invention. For example, other additives can be included, and the sulfated plant polysaccharide may be a mixture of two or more different plant-derived polysaccharides having at least one sulfate group. Further, the various methods and embodiments of the aspects disclosed herein can be included in combination with each other to produce variations of the disclosed methods and embodiments. Discussion of singular elements can include plural elements and vice-versa.

The order of steps can occur in a variety of sequences unless otherwise specifically limited. The various steps described herein can be combined with other steps, interlineated with the stated steps, and/or split into multiple steps. Similarly, elements have been described functionally and can be embodied as separate components or can be combined into components having multiple functions.

The inventions have been described in the context of preferred and other embodiments and not every embodiment of the invention has been described. Obvious modifications and alterations to the described embodiments are available to those of ordinary skill in the art. The disclosed and undisclosed embodiments are not intended to limit or restrict the scope or applicability of the invention conceived of by the Applicants, but rather, in conformity with the patent laws, Applicants intend to fully protect all such modifications and improvements that come within the scope or range of equivalent of the following claims.

What is claimed is:

1. A method of treating a glycocalyx-degradation associated disorder in a subject in need of such treatment, the method comprising:
administering an effective amount of pharmaceutical composition comprising a compound of formula (I), 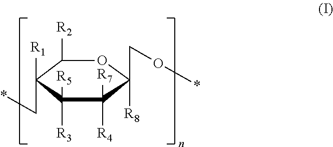
or a solvate, prodrug, hydrate, derivative, or analog thereof,
wherein,
R₁, R₂, R₃, R₄, R₅, R₆, R₇ and R₈ are either the same or different,
at least one of R₁-R₈ is hydroxyl,
at least one of R₁-R₈ is OSO₃⁻M⁺, with M being selected from the group consisting of Na, K, Li, or H,
and the remaining residues are independently selected from the following groups consisting of hydroxyl, thiol, halo, nitro, cyano, alkyl, alkenyl, alkynyl, sulfonamido, amino acid, amino acid esters, amino acid amides, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, hydroxamic acid, tetrazolyl, carbohydrate, fucan, sorbitan, sugar, or alditol; and
n is an integer selected from 1 to 20,000, inclusive, and
wherein the points of chirality (*) within each residue may be alpha (α), beta (β), or alternating alpha and beta between residues.

2. A method for treating a glycocalyx-related disorder in a patient, the method comprising the steps of:
(a) administering an effective amount of a pharmaceutical composition comprising:
(1) a pharmaceutically effective amount of a sulfated polysaccharide that is not of animal origin, or pharmaceutically acceptable salts, hydrates, solvates, or prodrugs thereof; and
(2) a pharmaceutically acceptable carrier, wherein the administration of the pharmaceutical formulation positively acts upon the glycocalyx in the patient.

3. The method of claim 2, wherein the pharmaceutical composition is an oral composition.

4. The method of claim 3, wherein the oral pharmaceutical composition is in the form of a pill, capsule, or tablet.

5. The method of claim 2, wherein the glycocalyx-related disorder is sickle cell anemia.

6. The method of claim 2, wherein the glycocalyx-related disorder is a thalassemia syndrome characterized by a lack of or a decreased synthesis of the globin chains of HbA.

7. The method of claim 2, wherein the sulfated polysaccharide is an inhibitor of an enzyme that degrades a glycocalyx in the patient.

8. A therapeutic composition comprising:
a compound of formula (I), 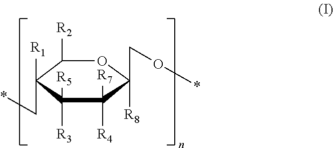
or a solvate, prodrug, hydrate, derivative, or analog thereof,
wherein,
R₁, R₂, R₃, R₄, R₅, R₆, R₇ and R₈ are either the same or different,
at least one of R₁-R₈ is hydroxyl,
at least one of R₁-R₈ is OSO₃⁻M⁺, with M being selected from the group consisting of Na, K, Li, or H,
and the remaining residues are independently selected from the following groups consisting of hydroxyl, thiol, halo, nitro, cyano, alkyl, alkenyl, alkynyl, sulfonamido, amino acid, amino acid esters, amino acid amides, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, hydroxamic acid, tetrazolyl, carbohydrate, fucan, sorbitan, sugar, or alditol; and
n is an integer selected from 1 to 20,000, inclusive, and
wherein the points of chirality (*) within each residue may be alpha (α), beta (β), or alternating alpha and beta between residues.

9. The therapeutic composition of claim 8, wherein the compound of formula (I) is a naturally-occurring compound that occurs naturally in a plant, algae, bacteria, or non-vertebrate organism.

10. A comestible composition comprising:
a compound of formula (I), 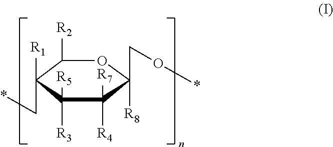
or a solvate, prodrug, hydrate, derivative, or analog thereof,
wherein,
R₁, R₂, R₃, R₄, R₅, R₆, R₇ and R₅ are either the same or different,
at least one of R₁-R₈ is hydroxyl,
at least one of R₁-R₈ is OSO₃⁻M⁺, with M being selected from the group consisting of Na, K, Li, or H,
and the remaining residues are independently selected from the following groups consisting of hydroxyl, thiol, halo, nitro, cyano, alkyl, alkenyl, alkynyl, sulfonamido, amino acid, amino acid esters, amino acid amides, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, hydroxamic acid, tetrazolyl, carbohydrate, fucan, sorbitan, sugar, or alditol; and
n is an integer selected from 1 to 20,000, inclusive, and
wherein the points of chirality (*) within each residue may be alpha (α), beta (β), or alternating alpha and beta between residues.

11. A method of screening for a therapeutic agent for a glycocalyx-related disease, the method which comprises:
a) contacting a test compound with cells expressing a heparin sulfate protein;
b) measuring expression levels of the protein in the cells; and
c) selecting a sulfated polysaccharide compound that increases the level of expression compared with that measured in the absence of the polysaccharide compound, thus selecting a therapeutic agent for the glycocalyx-related disease.

12. A method of suppressing glycocalyx degredation in endothelial cells in a region of a patient, the method comprising administering to the patient a composition comprising as an active ingredient a sulfated polysaccharide.

13. The method of claim 12, wherein the method has an effect of suppressing angiogenesis in cardiac valves.

14. The method of claim 12, wherein the method has an effect of suppressing vascular damage in a patient with diabetic retinopathy.

15. A method of treating a glycocalyx-associated disorder in a subject, the method comprising administering to a subject in need thereof a therapeutic amount of a composition comprising at least one purified, sulfated polysaccharide, sulfated polysaccharide polymer, or extract thereof, and wherein the composition is at a dose sufficient to displace heparans from at least one glycocalyx of an endothelium of the subject.

16. The method of claim 15, wherein the sulfated polysaccharide is rhamnan sulfate, or a rhamnan sulfate isolate.

17. The method of claim 16, wherein the rhamnan sulfate or rhamnan sulfate isolate is derived from the species Monostroma nitidum.

18. A therapeutic dosage form useful for the treatment of a vascular or vessel-related disorder in a subject, comprising a sulfated polysaccharide, a sulfated polysaccharide isolate of a natural organism, or an extract or polymer extract of a natural sulfated polysaccharide.

19. A method of treating metabolic syndrome, or one or more symptoms of metabolic syndrome in a subject, the method comprising administering to a subject in need thereof a composition comprising an effective amount of a non-animal derived sulfated polysaccharide, or a pharmaceutically acceptable salt, hydrate, solvate, or prodrug thereof.

20. A composition for the prevention and/or treatment of metabolic syndrome, which comprises at least one compound selected from among the compound represented by the formula I, formula II, or formula III, or a salt, solvate, hydrate, prodrug, or ester thereof, the content thereof being not lower than 0.0000001% by weight but lower than 100% by weight.
